SEQUENCE LISTING (36 sequences)

SEQ ID NO: 1 - protein, 9 aa, Homo sapiens
KTWGQYWZY

SEQ ID NO: 2 - protein, 9 aa, Homo sapiens
ITDQVPPSV

SEQ ID NO: 3 - protein, 9 aa, Homo sapiens
YLEPGPVTA

SEQ ID NO: 4 - protein, 10 aa, Homo sapiens
LLDGTATLRL

SEQ ID NO: 5 - protein, 10 aa, Homo sapiens
VLYRYGSFSV

SEQ ID NO: 6 - protein, 9 aa, Homo sapiens
AAGIGILTV

SEQ ID NO: 7 - protein, 9 aa, Homo sapiens
ILTVILGVL

SEQ ID NO: 8 - protein, 9 aa, Homo sapiens
MSLQRQFLR

SEQ ID NO: 9 - protein, 9 aa, Homo sapiens
MLLAVLYCL

SEQ ID NO: 10 - protein, 9 aa, Homo sapiens
YMNGTMSQV

SEQ ID NO: 11 - protein, 9 aa, Homo sapiens
SEIWRDIDF

SEQ ID NO: 12 - protein, 9 aa, Homo sapiens
AFLPWHRLF

SEQ ID NO: 13 - protein, 15 aa, Homo sapiens
QNILLSNAPLGPQFP

SEQ ID NO: 14 - protein, 13 aa, Homo sapiens
SYLQDSDPDSFQD

SEQ ID NO: 15 - protein, 9 aa, Homo sapiens
EADPTGHSY

SEQ ID NO: 16 - protein, 9 aa, Homo sapiens
SAYGEPRKL

SEQ ID NO: 17 - protein, 9 aa, Homo sapiens
EVDPIGHLY

SEQ ID NO: 18 - protein, 9 aa, Homo sapiens
FLWGPRALV

SEQ ID NO: 19 - protein, 9 aa, Homo sapiens
AARAVFLAL

SEQ ID NO: 20 - protein, 8 aa, Homo sapiens
YRPRPRRY

SEQ ID NO: 21 - protein, 9 aa, Homo sapiens
VLPDVFIRC

SEQ ID NO: 22 - protein, 9 aa, Homo sapiens
AYGLDFYIL

SEQ ID NO: 23 - protein, 9 aa, Homo sapiens
YLSGANLNL

SEQ ID NO: 24 - protein, 9 aa, Homo sapiens
YLSGADLNL

SEQ ID NO: 25 - protein, 9 aa, Homo sapiens
SYLDSGIHF

SEQ ID NO: 26 - protein, 9 aa, Homo sapiens
EEKLIVVLF

SEQ ID NO: 27 - protein, 10 aa, Homo sapiens
ACDPHSGHFV

SEQ ID NO: 28 - protein, 9 aa, Homo sapiens
IISAVVGIL

SEQ ID NO: 29 - protein, 9 aa, Homo sapiens
KIFGSLAFL

SEQ ID NO: 30 - protein, 10 aa, Homo sapiens
YMLDLQPETT

SEQ ID NO: 31 - protein, 20 aa, Homo sapiens
PDTRPAPGSTAPPAHGVTSA

SEQ ID NO: 32 - protein, 30 aa, Homo sapiens
FLTPKKLQCVDLHVISNDVCAQVHPQKVTK

SEQ ID NO: 33 - protein, 10 aa, Homo sapiens
FLTPKKLQCV

SEQ ID NO: 34 - protein, 9 aa, Homo sapiens
KLQCVDLHV

SEQ ID NO: 35 - protein, 10 aa, Homo sapiens
VISNDVCAQV

SEQ ID NO: 36 - protein, 9 aa, Homo sapiens
QVHPQKVTK

(19) United States
(12) Patent Application Publication
(10) Pub. No.: US 2004/0091995 A1
(43) Pub. Date: May 13, 2004

(54) Recombinant non-replicating virus expressing gm-csf and uses thereof to enhance immune responses

(76) Inventors: Jeffrey Schlom, Potomac, MD (US); John W. Greiner, Ijamsville, MD (US); Erik Kass, Chevy Chase, MD (US); Dennis Panicali, Acton, MA (US)

Correspondence Address: HELLER EHRMAN WHITE & MCAULIFFE LLP, 1666 K STREET,NW, SUITE 300, WASHINGTON, DC, 20006, US

(21) Appl. No.: 10/297,168

(22) PCT Filed: Jun. 15, 2001
(86) PCT No.: PCT/US01/19201

(51) Int. Cl. A61K 39/275; A61K 39/285; C12N 7/00; A61K 48/00

(52) U.S. Cl. 435/235.1; 424/232.1; 424/93.2

(57) ABSTRACT

Replication-defective recombinant poxvirus encoding granulocyte-macrophage colony-stimulating factor (GM-CSF) are disclosed for use in enriching an immunization site with antigen-presenting cells (APC), for enhancing an immunological response to antigen or immunological epitopes by functioning as a biological adjuvant, for prevention or treatment of neutropenia, and for the treatment of myeloidysplastic syndromes. Compositions comprising a replication-defective recombinant virus encoding GM-CSF alone or in combination with a recombinant virus encoding an antigen and optionally encoding an immunostimulatory molecule are disclosed for enhancing antigen-specific immunological responses, in particular enhancing tumor antigen responses for anti-tumor therapy. Methods for enriching an immunization site with APC and for enhancing immunological responses to an antigen or immunological epitope using replication-defective recombinant poxvirus encoding GM-CSF are disclosed. The superiority of the use of a replication-defective recombinant avian poxvirus encoding GM-CSF over the use of recombinant GM-CSF is described.

### FIELD OF THE INVENTION

The present invention relates to recombinant replication-defective virus expressing the cytokine, granulocyte-monocyte colony stimulating factor (GM-CSF) for use in enhancing immune responses and for treating neutropenia and myeloidysplastic syndromes. More specifically, the invention relates to recombinant replication-defective avian poxvirus expressing GM-CSF for use as a biological adjuvant for enhancing immune responses, in particular anti-tumor responses, and for treating neutropenia and myeloidysplastic syndromes and compositions comprising same.

### BACKGROUND OF THE INVENTION

By virtue of its actions as a major stimulatory cytokine for Langerhans and dendritic cells (1-3), GM-CSF⁵ is thought to function as a biological vaccine adjuvant. Experimental and clinical studies suggest that recombinant GM-CSF protein can boost host immunity directed at a variety of immunogens (4-14). In most of those studies, the recombinant GM-CSF protein (recGM-CSF) was administered for 4-5 consecutive days, beginning with co-injection with the antigen (15). Other approaches have delivered GM-CSF in DNA plasmids (16, 17), fusion proteins (18), retroviral vectors (19, 20) and replication competent vaccinia vectors (45), all of which have, for the most part, augmented host immunity. The use of the vaccinia-GM-CSF recombinant virus is questionable since repeated injections may be problematical (23) due to host anti-vector immune responses. Replication-defective avian poxviruses have been constructed to express cytokine gene products (24, 25) and shown herein to be more suitable to deliver GM-CSF to a site of immunization than prior art methods.

### SUMMARY OF THE INVENTION

An aspect of the invention is a composition comprising a recombinant replication-defective virus encoding GM-CSF, alone or in combination with a source of antigen or epitope source.

A further aspect of the invention is a composition comprising a recombinant replication-defective virus encoding both the GM-CSF and an antigen or immunological epitope thereof, in particular one or more tumor associated antigens.

Another aspect of the invention is a composition comprising a recombinant replication-defective virus encoding GM-CSF in combination with a vector expressing an antigen, alone or in combination with at least one immunostimulatory molecule.

An additional aspect of the invention is a composition comprising a recombinant replication-defective poxvirus encoding GM-CSF alone or in combination with a vector expressing at least one antigen or immunological epitope thereof, with or without a gene encoding at least one immunostimulatory molecule.

Another aspect of the invention is a composition comprising a recombinant avipox virus encoding GM-CSF alone or in combination with a vector expressing at least one tumor-associated antigen or immunological epitope thereof, with or without a gene encoding at least one immunostimulatory molecule.

One aspect of the invention is a composition comprising a recombinant replication-defective virus encoding GM-CSF in combination with a recombinant replication-defective virus expressing at least one antigen or immunological epitope thereof, with or without a gene encoding at least one immunostimulatory molecule.

Another aspect of the invention is a composition comprising a recombinant replication-defective avian poxvirus encoding GM-CSF in combination with a recombinant replication-defective avian poxvirus expressing at least one antigen or immunological epitope thereof.

Another aspect of the invention is a composition comprising a recombinant replication defective virus encoding GM-CSF in combination with an antibiotic, antifungal agent, anti-parasitic agent, anti-viral agent, or combination thereof.

Yet another aspect of the invention is a composition comprising a recombinant replication-defective virus encoding GM-CSF in combination with erythropoietin.

The invention further provides a composition comprising a recombinant replication-defective virus encoding GM-CSF in combination with a bispecific antibody.

The present invention provides host cells infected with a first vector of a recombinant replication-defective virus encoding GM-CSF molecules causing expression of the GM-CSF in the host cells. A second vector may further provide a foreign gene encoding at least one target antigen or immunological epitope thereof to the host cell, and/or foreign genes encoding one or more costimulatory molecules.

The present invention provides antigen-presenting cells (APCs) or tumor cells infected with a first vector of a recombinant replication-defective virus encoding GM-CSF causing expression of GM-CSF. A second vector may further provide a foreign gene encoding at least one target antigen or immunological epitope thereof to the host cell, and/or genes encoding one or more costimulatory molecules.

The present invention further provides host cells infected with a recombinant avipox virus causing expression of GM-CSF. The host cell may also be infected with a recombinant vector encoding at least one target antigen or immunological epitope thereof, and/or encoding at least one immunostimulatory molecule.

Another aspect of the invention is a dendritic cell (DC) and precursor thereof infected with a replication-defective virus encoding GM-CSF. The DCs and precursors thereof may further be engineered to express foreign genes encoding at least one target antigen or immunological epitope thereof, and/or engineered to express at least one immunostimulatory molecule.

Yet another aspect of the invention is a DC and precursors thereof genetically engineered to co-express GM-CSF and at least three exogenous costimulatory molecules. The DCs and precursor thereof may further be engineered to express foreign genes encoding at least one target antigen or immunological epitope thereof.

The present invention further provides a DC and precursors thereof genetically engineered to co-express GM-CSF, at least one B7 molecule, ICAM-1 and LFA-3. The DCs and precursor thereof may further be engineered to express foreign genes encoding at least one target antigen or immunological epitope thereof.

The invention further provides host cells infected with the recombinant replication-defective virus encoding GM-CSF as a source for commericial production of GM-CSF.

An object of the invention is to provide a method of enhancing an immune response to an antigen or epitope thereof comprising administration of a recombinant replication-defective virus expressing GM-CSF in an amount sufficient to enhance the immune response to the antigen or epitope thereof.

Another object of the invention is to provide a method of enhancing an immune response to an antigen or epitope thereof comprising administration of a recombinant replication-defective poxvirus expressing GM-CSF, alone or in combination with at least one antigen or immunological epitope source in an amount sufficient to enhance the immune response to the antigen or epitope thereof.

Another object of the invention is to provide a method of enhancing an immune response to at least one antigen or immunological epitope thereof comprising administration of a first recombinant vector encoding GM-CSF followed by administration of a second recombinant vector encoding GM-CSF, wherein at least one recombinant vector is a replication-defective virus.

A further object of the invention is to provide a method of enriching regional lymph nodes with antigen presenting cells (APCs) using recombinant replication-defective virus encoding GM-CSF.

The present invention further provides a method of generating antitumor immunity comprising administration of a recombinant replication-defective virus encoding GM-CSF, alone or in combination with at least one tumor antigen source, preferably a recombinant virus encoding at least one tumor antigen or immunological epitope thereof.

In another method of enhancing immunological responses, APCs or tumor cells infected with a recombinant replication-defective virus encoding GM-CSF are provided to a mammal in an effective amount to enhance immunological responses. The APC or tumor cell may further express foreign genes encoding at least one target antigen or immunological epitope thereof, alone or in combination with a gene encoding at least one costimulatory molecule for enhancement of immune responses. A target antigen or immunological epitope thereof may be administered to the mammal prior to, concurrently with or subsequent to the administration of the APC or tumor cell. In addition, or alternatively, APCs or tumor cells are pulsed with at least one target antigen or immunological epitope thereof prior to administration to the mammal.

Another object of the invention is to provide a method-for prevention or treatment of neutropenia using a recombinant replication-defective virus encoding GM-CSF.

A further object of the invention is to provide a method for treating myeloidysplastic syndromes using a recombinant replication-defective virus encoding GM-CSF in combination with erytropoietin.

Another aspect of the invention is a plasmid encoding GM-CSF for use in making a replication-defective virus encoding GM-CSF.

### BRIEF DESCRIPTION OF THE FIGURES

These and other objects, features and many of the attendant advantages of the invention will be better understood upon a reading of the detailed description of the invention when considered in connection with the accompanying drawings wherein:

FIG. 1. Shows plasmid vector pT5091 for generation of rF-muGM-CSF.

FIG. 2. Shows plasmid vector pT5052 for generation of rF-huGM-CSF.

FIG. 3. Shows plasmid vector pT5051 for generation of rV-huGM-CSF.

FIGS. 4A-4E. Shows the genomic structure of recombinant poxviruses expressing GM-CSF. BamHI J is the site of insertion in the fowlpox genome of the foreign genes. Hind III J or HindIII M is the site of insertion in the vaccinia virus genome. Deletion III is the site of insertion in the MVA genome. 40K, C1, P1 and P2 are poxviral promoters.

FIGS. 5A-5C. Shows the genomic structure of recombinant poxviruses co-expressing GM-CSF with a tumor-associate antigen (TAA). BamHI J is the site of insertion in the fowlpox genome of the foreign genes. Hind III J is the site of insertion in the vaccinia virus genome. Deletion III is the site of insertion in the MVA genome. P1, P2 and P3 are poxviral promoters.

FIG. 6. Production of murine GM-CSF by recombinant avipox-GM-CSF viruses. MC-38 cells were infected with of 5 MOI of either avipox(F)-GM-CSF, avipox(F)-WT, avipox(A)-GM-CSF or avipox(A)-RG as outlined in the Materials and Methods. Control cells received no virus. Cells were grown for 3 days and supernatants were collected every 24 hr. Murine GM-CSF levels were measured using the GM-CSF-dependent FDCP-1 indicator cell line and are presented as ng GM-CSF produced/10⁶ cells/24 h. Data are the mean ±SE from triplicate wells from a representative experiment that was repeated with similar results.

FIGS. 7A to 7D. MHC class II-expressing cells in regional lymph nodes of mice treated with recombinant avipox viruses expressing GM-CSF. In FIGS. 7A and 7B, groups of B6 mice (20-30 mice) were given a single s.c. injection (day 1, arrows) of either 10⁷ pfu (2A) or 10⁸ pfu (2B) avipox(F)-GM-CSF (closed circles) or avipox(F)-WT (open circles). In FIGS. 7C and 7D, B6 mice received avipox(A)-GM-CSF (closed circles) or avipox(A)-RG (open circles) at 10⁷ pfu and 10⁸ pfu, respectively. Other B6 mice (n=20) received daily injections of 20 μg recGM-CSF (FIG. 7A, dashed line, closed triangles) for four days (solid horizontal line). Control mice (open triangles, all panels) received 100 μl HBSS. Mice (4-6/group) were sacrificed at each time point, inguinal lymph nodes were removed, combined and the total lymph node cells were counted using a hemocytometer. The percentage of class II-expressing cells was determined by I-Ab⁺ cells using flow cytometry and the total number of class II-expressing cells was calculated by: Total lymph node cells multiplied by % class II⁺ cells=total class II⁺ cells. Data represent the results from 3-4 experiments in which each time point was the average of 2-3 separate determinations (SD=<15%).

FIG. 8. Total number of APC per regional lymph node in mice treated with avipox-GM-CSF or recGM-CSF. B6 mice (8-12/group) were administered 10⁸ pfu (indicated by the arrow) of avipox(F)-GM-CSF (solid triangles), avipox(F)-WT (open triangles), avipox(A)-GM-CSF (solid squares) or avipox(A)-rabies glycoprotein (RG) (open squares) as outlined in FIGS. 7A-7D. Recombinant GM-CSF (20 μg, solid circles) was given to a cohort of mice (n=15) for four (4) consecutive days (solid horizontal line). Control mice (dashed line) received UBSS. Regional lymph nodes were removed at the indicated time points and the total number of CD11c⁺/I-Ab⁺ cells determined as summarized in the Materials and Methods. Data represent the mean ±SE of a composite of findings from 3-4 separate experiments in which each time point was examined 2-3 times.

FIGS. 9A-9B. Effects of avipox (FIG. 10A)-GM-CSF on the generation of an alloreactive CTL. Naive, B6 mice were treated with 10⁸ pfu of either avipox(A)-GM-CSF (closed triangles), avipox(A)-RG (closed squares), as previously described. Control mice received HBSS (closed circles). Seven (FIG. 9A) and 21 (FIG. 9B) days later, mice (3-4) were sacrificed, regional lymph nodes were removed and single cell suspensions were prepared. Those lymph node cells were irradiated (5000 rad) and used as APC. To setup a unidirectional MLC, a 1:1 ratio between responders (BALB/C splenocytes) and stimulators (irradiated B6 lymph node cells) was incubated in 10 ml medium in T-25 flasks for 5 days @37° C. The cells were harvested and cytotoxicity measured as described in the Materials and Methods. Cytolysis is shown for the allogenic H-2b target cells (MC-38), whereas, that for the syngeneic H-2d cells (P815) was <8% for all groups. Data represent the mean ±SE from quadruplicate determinations from a single experiment which was repeated with the same results.

FIGS. 10A and 10B. (A) Changes in lymph node class II-expressing cells following multiple injections of avipox(A)-GM-CSF or avipox(A)-RG. B6 mice (15/group) were injected with 10⁷ pfu of either avipox(A)-GM-CSF (solid circles), avipox(A)-RG (open circles) or HBSS (closed triangles). At each time point, 2-5 mice/group were sacrificed, bilateral inguinal nodes were isolated and the total class II expressing cells determined as outlined for FIGS. 7A-7D. Data represent the results from a single experiment. (FIG. 10B) Sera samples from mice were analyzed for the presence of anti-avipox(A) (hatched bars) or anti-GM-CSF IgG (solid bars) antibody titers (see Materials and Methods). Serum anti-avipox(A) and anti-GM-CSF antibody titers are the mean (SE<10%) from two individual mice from the avipox(A)-GM-CSF and untreated groups analyzed at each time point.

FIG. 11. Generation of anti-CEA IgG serum titers in CEA.Tg mice. CEA.Tg mice were vaccinated (2×) with avipox(A)-CEA alone (10⁸ pfu, panel B) or in combination with recGM-CSF (20 μg, panel C) or avipox(A)-GM-CSF (10⁸, pfu, panel D) as outlined in the Materials and Methods. Other mice received 2 injections of either avipox(A)-RG (10⁸ pfu) or HBSS (panel A). Two weeks after the final treatment, mice were bled and serum tested for the presence of anti-CEA IgG antibodies as previously described. Data represent the serum antibody titers for individual mice. Titers which were <100 were considered negative.

FIGS. 12A-12B. Generation of CEA-₅₂₆₋₄₃₃ specific CTL responses in CEA.Tg mice vaccinated with avipox(A)-CEA in combination with avipox(A)-GM-CSF or recGM-CSF. CEA.Tg mice were vaccinated as outlined in the Materials and Methods. Purified splenic T cells were subjected to 3 rounds of in vitro stimulation in the presence of 10 units IL-2 and 1 μg CEA peptide/ml. T cell lines grew from those CEA.Tg mice vaccinated with avipox(A)-CEA alone (◯, ●), avipox(A)-CEA+avipox(A)-GM-CSF (▪, □) and avipox(A)-CEA+recGM-CSF (▴, Δ). Cytolytic activity was tested against EL4 targets cells incubated in the presence of 0.2 μg of CEA₅₂₆₋₅₂₂ (solid symbols) (FIG. 12A) or a control peptide (i.e., Flu NP₃₆₆₋₃₇₄) (open symbols). Those same T cell lines were incubated with freshly isolated, irradiated APC and 0.01-1 μg CEA peptide for 48 hr and supernatants collected. IFN-γ production was determined by ELISA (FIG. 12B). Data are the mean ±SEM from three separate wells in a representative experiment that was repeated with similar results. No detectable IL-4 levels were found.

FIGS. 13A-13D. Antitumor immunity in CEA.Tg mice vacciniated with avipox (A)-CEA combined with either avipox (A)-GM-CSF (13A) or rGM-CSF (13B). FIGS. 13A and 13B, the growth of MC-38-CEA-2 tumors in individual CEA.Tg mice that were vaccinated with avipox-CEA combined with either avipox (A)-GM-CSF (13A, arrow) or rGM-CSF (13B, arrow, solid horizontal line). N, number of tumor-free mice at day 56. Data are combined with two separate experiments. Solid lines in FIGS. 13A and 13B indicate mice in which tumor regression was observed. FIG. 13C, survival of the CEA-Tg mice vaccinated with avipox (A), CEA.Tg mice vaccinated with avipox (A)-CEA alone (●), or combined with avipox (A)-GM-CSF (▪) or rGM-CSF (▴). Untreated CEA.Tg mice (dashed line) received HBSS. Vaccination with avipox (A)-RG alone or combined with either avipox (A)-GM-CSF or rGM-CSF did not alter overall survival (data not shown). FIG. 13D. CEA.Tg mice that rejected MC-38-CEA-2 tumors after being vaccinated with avipox (A)-CEA and avipox (A)-GM-CSF (n=5, ▴) or avipox (A)-CEA+rGM-CSF (n=4; ●) were challenged with 3×10⁵ MC38-CEA-2 tumor cells. Dashed line, survival of 10 naive CEA.Tg mice that were administered the same tumor dose.

FIGS. 14A-14C. Avipox(F)-GM-CSF enhances CEA-specific T-cell responses to CEA vaccines. FIG. 14A: T-cell responses from mice vaccinated with buffer (closed boxes), avipox(F)-WT (closed diamonds), or avipox (F)-WT plus avipox(F)-GM-CSF (closed circles). FIG. 14B: T-cell responses from mice vaccinated with avipox (F)-CEA (closed diamonds), or avipox(F)-CEA plus avipox(F)-GM-CSF (closed circles). FIG. 14C: T-cell responses from mice vaccinated with avipox(F)-CEA/TRICOM (i.e. avipox expressing CEA, B7.1, ICAM-1, and LFA-3; closed diamonds), or avipox(F)-CEA/TRICOM plus avipox(F)-GM-CSF (closed circles). Inserts in each panel demonstrate that T-cell responses to Con A (positive control) and ovalbumin (negative control) were the same for all groups.

FIG. 15. Lymphoproliferative response to the whole protein, β-galactosidase (β-gal) by splenocytes isolated from mice immunized with β-gal combined with incomplete Freund's adjuvant (IFA with or without fowlpox murine GM-CSF (Fp-mu GM-CSF). (=untreated; ▾=β-gal+EFA; ♦=β-gal+IFA+Fp-muGM-CSF 10⁷ pfu; ▪=β-gal+Fp-WT).

### DETAIL DESCRIPTION OF TIE INVENTION

The present invention is a recombinant replication-defective virus encoding GM-CSF for use in enhancing immunological responses to an antigen or immunological epitope thereof. Recombinant replication-defective virus for use in the present invention include but are not limited to replication-defective poxvirus, herpes virus, adenovirus, adeno-associated virus (AAV) and other vectors incapable of replicating in mammalian cells, preferably human cells. In particular, the present invention is a recombinant replication-defective avian poxvirus, including fowlpox, canary pox virus and Modified Vaccinia Ankara strain (MVA) encoding GM-CSF for use as a biological adjuvant in enhancing immunological response to an antigen.

The recombinant replication-defective virus encoding GM-CSF of the present invention has utility in providing enhanced immunological response to cells of the immune system including antigen-presenting cells (APCs), T lymphocytes, B lymphocytes, NK cells and the like. The immunological response may be a generalized immune enhancing or upregulating effect as demonstrated by increased cytokine release, increase proliferation by immune cells, increased mitogen responsiveness and the like. Of particular interest is the immigration and enrichment of APCs at an immunological site caused by administration of the recombinant replication-defective virus encoding GM-CSF. The recombinant replication-defective virus encoding GM-CSF is a biological adjuvant in that it functions to increase APCs at the injection site. The recombinant replication-defective virus encoding GM-CSF may be used in combination with an antigen source for enhancement of antigen specific immunological responses. Such responses may include a cellular and/or a humoral response directed to a specific antigen or epitope thereof.

The recombinant-replication defective virus encoding GM-CSF provides an enhanced immunological response and advantages which are superior to those of natural GM-CSF protein, recombinant GM-CSF protein, GM-CSF-DNA plasmids, GM-CSF-fusion proteins, retroviral vectors encoding GM-CSF and vaccinia vectors encoding GM-CSF. The enhancement provided by the recombinant replication defective virus encoding GM-CSF is manifest both in the magnitude of the immune response and in the duration of the immune response.

Of particular interest are recombinant replication-defective fowlpox viruses and recombinant replication-defective canary pox viruses for delivery of a gene encoding GM-CSF to a host cell.

Construction of recombinant replication-defective fowlpox virus encoding GM-CSF is disclosed herein. Construction of a recombinant canarypox virus encoding GM-CSF is disclosed in Human Gene Therapy 1998 November:9(17):2481-92.

The recombinant replication-defective virus of the present invention comprises the gene encoding full length human GM-CSF (Gen Bank No. M1O663) or a mammalian gene encoding GM-CSF.

The present invention encompasses compositions, preferably pharmaceutically acceptable compositions comprising at least one recombinant replication-defective virus encoding GM-CSF alone or in combination with a source of antigen or epitope thereof. The composition may further comprise a conventional adjuvant. Sources of antigen or immunological epitopes thereof include but are not limited to proteins, peptides, lipids, lipoproteins, carbohydrates, polysaccharides, lipopolysaceharides, cells, cell fragments, cell extracts, antibodies, anti-idiotypic antibodies, apoptotic bodies and the like. The antigen or epitope source may be isolated from naturally occurring sources, chemically synthesized or genetically produced. A source of genetically produced antigen or epitope thereof include vectors encoding at least one antigen or epitope thereof, and the like. Cell sources of antigen or an immunological epitope thereof include but are not limited to bacteria, fungi, yeast, protozoans, virus, tumor cells, APCs, dendritic cells (DC), DC-tumor cell fusions and the like, as well as cells transfected or transduced with a gene encoding at least one antigen or epitope thereof.

In one particular embodiment, the antigen source is provided by one or more genes encoding one or more antigens or immunologically epitopes thereof, incorporated into the recombinant replication-defective virus encoding GM-CSF, for coexpression of the one or more antigens along with GM-CSF. Of particular interest are genes encoding tumor antigens or tumor-associated antigens.

In another embodiment, the composition comprises a recombinant replication-defective avipox virus encoding GM-CSF and an antigen source alone or in combination with a conventional adjuvant.

The present invention encompasses compositions, preferably pharmaceutically acceptable compositions comprising at least one recombinant replication-defective virus encoding GM-CSF, alone or in combination with at least one vector encoding an antigen or epitope thereof, and/or encoding one or more immunostimulatory molecules and a pharmaceutically acceptable carrier.

In another embodiment, the composition comprises a recombinant replication-defective avipox virus encoding GM-CSF in combination with a vector encoding at least one antigen or immunological epitope thereof. The vector for use in providing the gene(s) encoding the antigen or immunological epitope thereof having utility in the present invention include any vector capable of causing functional expression of one or more gene products in a mammalian host cell, preferably a human cell. Vectors useful in providing genes encoding the antigen include but are not limited to viral vectors, nucleic acid based vectors and the like, including but not limited to poxvirus, Herpes virus, adenovirus, alphavirus, retrovirus, picomavirus, iridovirus and the like. Poxviruses having utility in providing genes encoding antigens and/or genes encoding immunostimulatory molecules include replicating and non-replicating vectors.

In one embodiment, the composition comprises a recombinant replication-defective fowlpox encoding GM-CSF in combination with a recombinant fowlpox encoding at least one antigen or epitope thereof alone or in combination with a gene encoding one or more costimulatory molecules. In another embodiment, the composition comprises a recombinant replication-defective avipox encoding GM-CSF in combination with a recombinant replication-defective avipox encoding at least one antigen and encoding a B7 molecule. In another embodiment the recombinant replication-defective avipox virus encoding at least one antigen also encodes multiple costimulatory molecules such as B7/LFA-3/ICAM-1. The magnitude of the immune response to the antigen, epitope, or cells expressing the antigen resulting from administration of the composition of the present invention is significantly greater than that achieved using recGM-CSF in combination with a recombinant virus encoding an antigen.

The target antigen, as used herein, is an antigen or immunological epitope on the antigen which is crucial in immune recognition and ultimate elimination or control of the disease-causing agent or disease state in a mammal. The immune recognition may be cellular and/or humoral. In the case of intracellular pathogens and cancer, immune recognition is preferably a T lymphocyte response.

Target antigen includes an antigen associated with a preneoplastic or hyperplastic state. Target antigen may also be associated with, or causative of cancer. Such target antigen may be a tumor cell, tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D (46) and the like (GenBank Accession No. M29540), MART-1 (Kawakami et al, J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987, TRP-1 (gp75) (Cohen et al Nucleic Acid Res. 18:2807-2808, 1990; U.S. Pat. No. 5,840,839), NY-ESO-1 (Chen et al PNAS 94: 1914-1918, 1997), TRP-2 (Jackson et al EMBO J, 11:527-535, 1992), TAG72, KSA, CA-125, PSA, HER-2/neu/c-erb/B2, (U.S. Pat. No. 5,550,214), BRC-I, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each.

Possible human tumor antigens and tissue specific antigens as well as immunological epitopes thereof for targeting using the present invention include but are not limited to those exemplified in Table 1.

TABLE 1
Antigens and Epitopes Recognized by T Cells
Human target tumor antigens recognized by T cells

| Target | | Immunological | |
|---|---|---|---|
| antigens | Restriction element | Peptide epitope | SEQ. ID No. |
| gp 100 | HLA-A2 | KTWGQYWZY | 1 |
| | HLA-A2 | ITDQVPPSV | 2 |
| | HLA-A2 | YLEPGPVTA | 3 |
| | HLA-A2 | LLDGTATLRL | 4 |
| | HLA-A2 | VLYRYGSFSV | 5 |
| MART1-/Melan A | HLA-A2 | AAGIGILTV | 6 |
| | HLA-A2 | ILTVILGVL | 7 |
| TRP-1 (GP75) | HLA-A31 | MSLQRQFLR | 8 |
| Tyrosinase | HLA-A2 | MLLAVLYCL | 9 |
| | HLA-A2 | YMNGTMSQV | 10 |
| | HLA-B44 | SEIWRDIDF | 11 |
| | HLA-A24 | AFLPWHRLF | 12 |
| | HLA-DR4 | QNILLSNAPLGPQFP | 13 |
| | HLA-DR4 | SYLQDSDPDSFQD | 14 |
| MAGE-1 | HLA-A1 | EADPTGHSY | 15 |
| | HLA-Cw16 | SAYGEPRKL | 16 |
| MAGE-3 | HLA-A1 | EVDPIGHLY | 17 |
| | HLA-A2 | FLWGPRALV | 18 |
| BAGE | HLA-Cw16 | AARAVFLAL | 19 |
| GAGE-1,2 | HLA-Cw6 | YRPRPRRY | 20 |
| N-acetylglucos- | HLA-A2 | VLPDVFIRC | 21 |
| aminyltransferase-V | | | |
| p15 | HLA-A24 | AYGLDFYIL | 22 |
| CEA | | YLSGANLNL(CAP1) | 23 |
| | | YLSGADLNL (CAP1-6D) | 24 |
| β-catenin | HLA-A24 | SYLDSGIHF | 25 |
| MUM-1 | HLA-B44 | EEKLIVVLF | 26 |
| CDK4 | HLA-A2 | ACDPHSGHFV | 27 |
| HER-2/neu | HLA-A2 | LISAVVGIL | 28 |
| (Breast and ovarian | HLA-A2 | KIFGSLAFL | 29 |
| carcinoma) | | | |
| Human papillomavirus- | HLA-A2 | YMLDLQPETIT | 30 |
| E6,E7 | | | |
| (cervical carcinoma) | | | |
| MUC-1 | Non-MHC restricted | PDTRPAPGSTAPPAHGVTSA | 31 |
| | MHC restricted (and portions thereof) | | |
| (Breast, ovarian and | A2, A3 | FLTPKKLQCVDLHVISNDVCA- | 32 |
| pancreatic carcinoma) | | | |
| PSA | | QVHPQKVTK | |
| | | FLTPKKLQCV | 33 |
| | | KLQCVDLHV | 34 |
| | | VISNDVCAQV | 35 |
| | | QVHPQKVTK | 36 |

The target antigen may be cell associated, derived or isolated from a pathogenic microorganism such as viruses including HIV, (Korber et al eds HIV Molecular Immunology Database, Los Alamos National Laboratory, Los Alamos, New Mex. 1977) influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV) and the like.

Target antigen may be cell associated, derived or isolated from pathogenic bacteria such as from Chlamydia (U.S. Pat. No. 5,869,608), Mycobacteria, Legionella, Meningiococcus, Group A Streptococcus, Sallnonella, Listeria, Hemophilus influenzae (U.S. Pat. No. 5,955,596) and the like.

Target antigen may be cell associated, derived or isolated from pathogenic yeast including Aspergillus, invasive Candida (U.S. Pat. No. 5,645,992), Nocardia, Histoplasmosis, Cryptosporidia and the like.

Target antigen may be cell associated, derived or isolated from a pathogenic protozoan and pathogenic parasites including but not limited to Pneumocystis carinii, Trypanosoma, Leislmania (U.S. Pat. No. 5,965,242), Plasmodium (U.S. Pat. No. 5,589,343) and Toxoplasma gondii.

Immunostimulatory molecules as used herein include but are not limited to the costimulatory molecules: B7, ICAM-1, LFA-3, 4-1BBL, CD59, CD40, CD70, VCAM-1, OX-40L and the like, as well as cytokines and chemokines including but not limited to IL-2, TNFα, IFNγ, IL-12, RANTES, MIP-1α, Flt-3L (U.S. Pat. Nos. 5,554,512; 5,843,423) and the like.

The gene sequence of murine B7.1 is disclosed in Freeman et al (J. Immunol. 143:2714-2722, 1989) and in GENBANK under Accession No. X60958. The gene sequence of murine B7.2 is disclosed in Azuma et al (Nature 366:76-79, 1993) and in GENBANK under Accession No. L25606 and MUSB72X.

The human homolog of the murine B7 costimulatory molecules include CD80, the homolog of murine B7.1, and CD86, the homolog of B7.2. The gene sequence of human B7.1 (CD80) is disclosed in GENBANK under Accession No. M27533, and the gene sequence of human B7.2 (CD86) is disclosed under Accession No. U04343 and AF099105.

The gene for murine ICAM-1 is disclosed in GenBank under Accession No. X52264 and the gene for the human ICAM-1 homolog, (CD54), is disclosed in Accession No. J03132.

The gene for murine LFA-3 is disclosed in GenBank under Accession No. X53526 and the gene for the human homolog is disclosed in Accession No. Y00636.

The gene for the murine 4-1BBL is disclosed in GenBank under Accession No. U02567. The gene for the human homolog, hu4-1BBL is disclosed in GenBank under Accession No. U03397.

The immunostimulatory molecules may be provided by a recombinant vector encoding the immunostimulatory molecule alone, or in combination with a nucleic acid sequence encoding a target antigen. In another embodiment, the composition provides recombinant vector encoding a target antigen and encoding the multiple costimulatory molecules B7/ICAM-1/LFA′-3 (TRICOM) in combination with a recombinant replication-defective virus encoding GM-CSF.

A conventional adjuvant as used herein includes but is not limited to alum, Ribi DETOX™, Freund's adjuvant, Freund's complete adjuvant, QS21 and the like.

Diseases may be treated or prevented by use of the present invention and include diseases caused by viruses, bacteria, yeast, parasites, protozoans, cancer cells and the like. The recombinant replication-defective virus encoding GM-CSF may be used as a generalized immune enhancer and as such has utility in treating diseases of no known etiological cause.

Preneoplastic or hyperplastic states which may be treated or prevented using a recombinant replication-defective virus encoding GM-CSF of the present invention include but are not limited to preneoplastic or hyperplastic states such as colon polyps, Crohn's disease, ulcerative colitis, breast lesions and the like.

Cancers which may be treated using the recombinant replication-defective virus encoding GM-CSF of the present invention include but are not limited to primary or metastatic melanoma, adenocarcinoma, squamous cell carcinoma, adenosqtamous cell carcinoma, thymoma, lymphoma, sarcoma, lung cancer, liver cancer, non-Hodgkins lymphoma, Hodgkins lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

Several uses of recombinant replication-defective virus encoding GM-CSF are outlined in Table 2.

TABLE 2
Uses of the Recombinant Replication-Defective Virus Encoding GM-CSF
A. Adjuvant

| | |
|---|---|
| I. | With an Antigen (Ag)-protein, peptide, cell extract, etc., carbohy- |
| | drate, Ab, anti-is Ab |
| | all +/− conventional adjuvant (Freund's complete adjuvant, |
| | Freund's incomplete adjuvant, Ribi Detox ™, Alum, QS-21) |
| | (a) Ag + rRDV-GM-CSF¹ |
| | r vector-Ag + rRDV-GM-CSF |
| | rAvipox vector-Ag + rRDV-GM-CSF |
| | (b) any recombinant vector encoding Ag + B7 + rAvipox-GM-CSF |
| | any recombinant vector encoding Ag + TRICOM + |
| | rAvipox-GM-CSF |
| | any recombinant vector encoding Ag + rAvipox-GM-CSF |
| | rAvipox-Ag-one or more costimulatory B7 + rAvipox-GM-CSF |
| | rAvipox-Ag-TRICOM + rAvipox-GM-CSF |
| II. | Direct Tumor Injection In Situ |
| | malenoma or breast skin lesions, and the like |
| | at surgery, e.g. colorectal, pancreatic cancer |
| | rRDV-GM-CSF ± r vector-B7 or r vector-TRICOM; ± another |
| | cytokine such as IL-12 |
| III. | Intra Lymph Node Injection |
| | either distal or draining tumor site |
| | rRDV-GM-CSF alone |
| | all those noted in I(a) and (b) |
| IV. | Infection of Tumor Cells Ex-Vivo For Use as a Vaccine |
| | tumor cells can be from the same patient (autologous) or a cell |
| | line(s) from different patients (allogeneic) |
| | infect with rRDV-GM-CSF |
| | infect with rRDV-GM-CSF + r vector-B7 |
| | infect with rRDV-GM-CSF + r vector-TRICOM |
| | infect with tRDV-GM-CSF + r vector-IFN (gamma or alpha) |
| | infect with rRDV-GM-CSF + r vector-any cytokine |
| V. | Infection of Dendritic Cells (DC) ex vivo for a vaccine to be in- |
| | jected s.c., i.d., or i.v. |
| | (a) Pulse DC with peptide, protein, Ab, cell extract, apoptic bodies |
| | and the like. |
| | Infect DC with rRDV-GM-CSF |
| | rRDV-GM-CSF + r vector-B7 |
| | rRDV-GM-CSF + r vector-TRICOM |
| | (b) Infect DC with: |
| | rRDV-GM-CSF-Ag |
| | rRDV-GM-CSF + r vector-Ag |
| | rRDV-GM-CSF + r vector-Ag-B7 |
| | rRDV-GM-CSF + r vector-Ag-TRICOM |
| | rAvipox-GM-CSF + rAvipox |
| | rAvipox-GM-CSF + rAvipox-B7 |
| | rAvipox-GM-CSF + rAvipox-TRICOM |
| V. | Infect DC-Tumor Cell Fusion Product with: |
| | rRDV-GM-CSF |
| | rRDV-GM-CSF + r vector-B7 |
| | rRDV-GM-CSF + r vector-TRICOM |

B-Treatment of neutropenia

rRDV-GM-CSF

C-Treatment of Myeloidysplastic syndromes

rRDV-GM-CSF + EPO
¹= Recombinant Replication Defective Virus Encoding GM-CSF = rRDV-GM-CSF

The present invention provides methods of enhancing immune responses using a recombinant replication-defective virus encoding GM-CSF for recruitment of antigen presenting cells into an injection site. Moreover, the method provides enrichment of regional lymph nodes with antigen presenting cells.

The methods of the present invention provides enhancement of immune responses to a target antigen or epitope thereof.

The present invention also encompasses methods of treatment or prevention of a disease caused by pathogenic microorganisms or by cancer using a recombinant replication-defective virus encoding GM-CSF alone or in combination with an antigen source.

In the method of treatment, the administration of the recombinant vector of the invention may be for either “prophylactic” or “therapeutic” purpose. When provided prophylactically, the recombinant replication-defective virus encoding GM-CSF of the present invention is provided in advance of any symptom alone or prior to concurrently or preceding the administration of an antigen source. The prophylactic administration of the recombinant vector serves to prevent or ameliorate any subsequent infection or disease. When provided therapeutically, the recombinant replication-defective virus encoding GM-CSF is provided at or after the onset of a symptom of infection or disease. Thus the present invention may be provided either prior to the anticipated exposure to a disease-causing agent or disease state or after the initiation of the infection or disease.

The term “unit dose” as it pertains to the inoculum refers to physically discrete units suitable as unitary dosages for mammals, each unit containing a predetermined quantity of recombinant vector calculated to produce the desired adjuvant and immunogenic effect in association with the required diluent. The specifications for the novel unit dose of an inoculum of this invention are dictated by and are dependent upon the unique characteristics of the recombinant replication-defective virus encoding GM-CSF and the particular adjuvant and immunologic effect to be achieved.

The inoculum is typically prepared as a solution in tolerable (acceptable) diluent such as saline, phosphate-buffered saline or other physiologically tolerable diluent and the like to form an aqueous pharmaceutical composition.

The route of inoculation may be scarification, intravenous (I.V.), intramuscular (I.M.), subcutaneous (S.C.), intradermal (I.D.), intraperitoneal (I.P.), intratumor, topical, intranodal, intranasal, intraarterial, intravesical, and the like, which results in migration of APC into the injection site and regional lymph nodes and upregulation of APC functions to enhance an immune response against the disease causing agent. The dose is administered at least once. Subsequent doses may be administered as indicated.

In one example, the host is immunized at least once with a recombinant replication-defective virus encoding GM-CSF to elicit optimal concentration of APCs at a target site. Subsequent immunizations are provided with one or more antigens or epitopes sources. In another example, the host is first immunized with an antigen source such as proteins, peptides, polysaccharides, lipids, lipoproteins, lipopolysaccharides, antibodies, anti-idiotypic antibodies, cells, cell fragments, cell extracts, apoptotic bodies, attenuated or inactivated virus and the like, followed by administration of a recombinant replication-defective virus encoding GM-CSF. In another embodiment of the method, the recombinant replication-defective virus encoding GM-CSF is administered concurrently with an antigen or epitope source. A conventional adjuvant may optionally be provided.

In another embodiment, the host is immunized at least one with a recombinant replication-defective virus encoding GM-CSF as a primary dose. Boosting doses may comprise any recombinant vector encoding GM-CSF, preferably a recombinant virus encoding GM-CSF. The second recombinant vector encoding GM-CSF may be replication-competent or replication-defective. In one example, the priming dose is provided by replication-defective recombinant avipox virus encoding GM-CSF followed by a boosting dose of replication-competent recombinant vaccinia virus encoding GM-CSF. Such heterologous prime-boost regimes minimizes or reduces host anti-vector immune responses as are known in the art with multiple injections of recombinant vaccinia vectors. Variations in the prime-boost method are encompassed within the invention. For example, a replication-competent vector encoding GM-CSF may be provided as a priming dose, followed by one or more injections of a replication-defective virus encoding GM-CSF. The vectors may also provide a gene encoding one or more antigens, with or without a gene encoding one or more immunostimulatory molecules.

The recombinant replication-defective virus encoding GM-CSF may be provided in combination with a vaccine including but not limited to the standard childhood vaccines such as Diphtheria-Tetanus-Pertusis (DPT), Tetanus-Diphtheria (Id), DtaP, Haemophilus influenza type b (Hib) vaccine, DTaP-Hib vaccine, DTaP-IPV-Hib vaccine, mumps-measles-rubella (MMR) vaccine, as well as vaccines such as Hepatitis A vaccine, Hepatitis B vaccine, Lyme's disease vaccine, influenza vaccine, meningococcal polysaccharide (tetravalent A, C, W135 and Y), pneumococcal polysaccharide vaccine (23 valent), anthrax vaccine, cholera vaccine, plague vaccine, yellow fever vaccine, Bacillus Calmette-Guerin vaccine and the like.

In providing a mammal with the recombinant vector of the present invention, preferably a human, the dosage of administered recombinant vector will vary depending upon such factors as the mammal's age, weight, height, sex, general medical condition, previous medical history, disease progression, tumor burden and the like. In general, it is desirable to provide the recipient with a dosage of recombinant replication-defective virus encoding GM-CSF in the range of about 10⁵ to about 10¹⁰ plaque forming units per mammal, preferably a human, although a lower or higher dose may be administered.

The genetic definition of tumor-associated and tumor-specific antigens allows for the development of targeted antigen-specific vaccines for cancer therapy. The recombinant replication-defective viruses encoding GM-CSF in combination with a recombinant vector encoding a tumor associated or tumor specific antigen is a powerful system to elicit a specific immune response in terms of prevention in individuals with an increased risk of cancer development (preventive immunization), to shrink tumors prior to surgery, to prevent disease recurrence after primary surgery (anti-metastatic vaccination), or to expand the number of cytotoxic lymphocytes (CTL) in vivo, thus improving their effectiveness in eradication of diffuse tumors (treatment of established disease). Autologous lymphocytes (CD8⁺), either cytotoxic T lymphocytes and/or CD4⁺ helper T cells or NK cells may be generated ex vivo to a particular tumor antigen and transferred back to the tumor bearing patient (adoptive immunotherapy) in combination with the recombinant replication-defective virus encoding GM-CSF, along with a tumor antigen source.

In cancer treatments, the recombinant replication-defective virus encoding GM-CSF can be introduced into a mammal either prior to any evidence of cancer or to mediate regression of the disease in a mammal afflicted with a cancer.

Depending on the disease or condition to be treated and the method of treatment, an antigen source such as a recombinant vector comprising a nucleic acid sequence encoding a target antigen or immunological epitope thereof may additionally comprise genes encoding one or multiple costimulatory molecules, preferably B7 or B7/ICAM-1/LFA-3. The target antigen or immunological epitope thereof may be provided by a host cell infected with the recombinant vector as or a tumor cell endogenously expressing a tumor associated antigen or epitope thereof. In the case in which a tumor associated antigen is absent, not expressed or expressed at low levels in a host cell, a foreign gene encoding an exogenous tumor associated antigen may be provided. Further, genes encoding several different tumor associated antigens may be provided.

The quantity of recombinant vector encoding one or more tumor associated antigens (TAAs) and optionally encoding multiple costimulatory molecules in conjunction with a recombinant replication-defective virus encoding GM-CSF to be administered is based on the titer of virus particles. A preferred range of the immunogen to be administered is 10⁵ to 10¹⁰ virus particles per mammal, preferably a human. If the mammal to be immunized is already afflicted with cancer or metastatic cancer, the vaccine can be administered in conjunction with other therapeutic treatments. Moreover, the recombinant replication-defective virus, itself, may encode one or more TAAs, along with encoding GM-CSF.

In one method of treatment, recombinant replication-defective virus encoding GM-CSF is administered in vivo to a patient with cancer and autologous cytotoxic lymphocytes or tumor infiltrating lymphocytes may be obtained from blood, lymph nodes, tumor and the like. The lymphocytes are grown in culture and target antigen-specific lymphocytes are expanded by culturing in the presence of specific target antigen and either antigen presenting cells or target antigen pulsed APCs. The target antigen-specific lymphocytes are then reinfused back into the patient.

After immunization the efficacy of the vaccine can be assessed by production of antibodies or immune cells that recognize the antigen, as assessed by specific lytic activity or specific cytokine production or by tumor regression. One skilled in the art would know the conventional methods to assess the aforementioned parameters.

In one embodiment of the method of enhancing antigen-specific T-cell responses, mammals, preferably humans, are immunized with recombinant replication-defective virus encoding GM-CSF in combination with an rF- or rV-HIV-1 epitope/B7-1/ICAM-1/LFA-3 construct. The efficacy of the treatment may be monitored in vitro and/or in vivo by determining target antigen-specific lymphoproliferation, target antigen-specific cytolytic response, cytokine production, clinical responses and the like.

The method of enhancing antigen-specific T-cell responses may be used for any target antigen or immunological epitope thereof. Of particular interest are tumor associated antigens, tissue specific antigens and antigens of infectious agents.

In addition to administration of the recombinant replication-defective virus encoding GM-CSF to the patient, other exogenous immunomodulators or immunostimulatory molecules, chemotherapeutic drugs, antibiotics, antifungal drugs, antiviral drugs and the like alone or in combination thereof may be administered depending on the condition to be treated. Examples of other exogenously added agents include exogenous IL-2, IL-6, alpha-, beta- or gamma-interferon, tumor necrosis factor, Flt-3L, cyclophosphamide, cisplatinum, gancyclovir, amphotericin B, 5 fluorouracil, leucovorin, CPT-11, and the like, and combinations thereof.

Recombinant avian poxviruses (avipox) that express GM-CSF were examined for their ability to produce biologically active GM-CSF in vivo. Recombinant fowl pox (F) and canarypox (ALVAC) viruses expressing GM-CSF were administered as single s.c injections, and the regional lymph nodes draining the injection site were examined for cellular, phenotypic and functional changes at different time points. Changes in the regional lymph nodes were compared with the administration of 4 daily doses of recGM-CSF. The results demonstrated that a single injection of either recombinant avipox-GM-CSF virus induced (i) lymphadenopathy and (ii) increased the total number of class II-expressing and professional APC (CD1 1 c⁺/I-Ab⁺) within the draining lymph nodes. When the lymph nodes from mice injected with avipox-GM-CSF were used in in vitro mixed lymphocyte cultures, higher levels of T-cell priming and more potent allospecific lysis resulted, indicating the presence of higher numbers of functional APC within those nodes. Time-course studies showed that the cellular/phenotypic and functional changes occurring within the regional nodes of mice injected with a recombinant avipox-GM-CSF virus were sustained for 21-28 days. Moreover, upon repeated injections (3×) of the avipox-GM-CSF recombinant virus, the total number of class II-expressing lymph node cells was increased after each injection, despite the presence of anti-avipox antibody titers in the mice sera.

The present invention also examined whether GM-CSF administered in a recombinant avipox virus or as a recombinant protein could function as a biological adjuvant in a vaccine protocol designed to generate host immunity to a self, tumor antigen. The self, tumor antigen was CEA, a Mᵣ80,000-200,000 glycoprotein, whose overexpression on a large percentage of human adenocarcinomas (colon, pancreatic, breast, lung) has made it an attractive target for immunotherapy (26, 27). Since no CEA homologue has been identified in rodents, mice expressing human CEA as a transgene (28-30) are being used to study different vaccine strategies (31). In the present invention, avipox-CEA immunized CEA.Tg mice developed CEA-specific cellular immunity which could be enhanced by the addition of GM-CSF either as a recombinant avipox virus or recombinant protein. Based on the relative potencies of the CEA-specific cellular responses in the CEA.Tg, a single injection of an avipox-GM-CSF viruses was a more potent biological adjuvant than multiple daily injections of recGM-CSF. In immunotherapeutic protocols, complete regression of CEA-positive tumors was observed in 30-40% of the CEA.Tg mice after immunization with avipox-CEA in combination with either avipox-GM-CSF or recGM-CSF. Furthermore, those tumor-free mice were protected from subsequent tumor challenged. The findings demonstrate for the first time that recombinant avipox viruses expressing GM-CSF can deliver sustained levels of GM-CSF to an immunization site and can be used in combination with a recombinant avipox virus expressing a relatively weak, self antigen to augment host immunity and generate enhanced antitumor immunity.

The recombinant replication-defective virus encoding GM-CSF of the present invention are useful in methods of stimulating an enhanced humoral response both in vivo and in vitro. Such an enhanced humoral response may be monoclonal or polyclonal in nature. The enhancement of a humoral response may be determined by increased activation, proliferation and/or cytokine secretion by CD4⁺ T cells, increased proliferation or antibody production by B cells, increased antibody dependent cellular toxicity (ADCC), increased complement-mediated lysis, and the like. Antibody elicited using the recombinant replication-defective virus encoding GM-CSF of the present invention are expected to be higher affinity and/or avidity and higher titer than antibody elicited by standard methods. The antibody elicited by methods using the recombinant replication-defective virus encoding GM-CSF may recognize immunodominant target epitopes or nondominant target epitopes.

This invention further comprises an antibody or antibodies elicited by immunization with the recombinant replication-defective virus encoding GM-CSF in combination with an antigen source of the present invention. The antibody has specificity for and reacts or binds with the target antigen or immunological epitope thereof of interest. In this embodiment of the invention the antibodies are monoclonal or polyclonal in origin.

Exemplary antibody molecules are intact immunoglobulin molecules, substantially intact immunoglobulin molecules or those portions of an immunoglobulin molecule that contain the antigen binding site, including those portions of immunoglobulin molecules known in the art as F(ab), F(ab′), F(ab′)₂ and F(v). Polyclonal or monoclonal antibodies may be produced by methods known in the art. (Kohler and Milstein (1975) Nature 256, 495497; Campbell “Monoclonal Antibody Technology, the Production and Characterization of Rodent and Human Hybridomas” in Burdon et al. (eds.) (1985) “Laboratory Techniques in Biochemistry and Molecular Biology,” Volume 13, Elsevier Science Publishers, Amsterdam). The antibodies or antigen binding fragments may also be produced by genetic engineering. The technology for expression of both heavy and light chain genes in E. coli is the subject of the PCT patent applications: publication number WO 901443, WO 901443 and WO 9014424 and in Huse et al. (1989) Science 246:1275-1281.

In one embodiment the antibodies of this invention are used in immunoassays to detect the novel antigen of interest in biological samples.

In one embodiment, the antibodies of this invention generated by immunization with a recombinant replication-defective virus encoding GM-CSF in combination with a recombinant virus expressing a TAA and expressing B7-1, ICAM-1 and LFA-3 are used to assess the presence of the a TAA from a tissue biopsy of a mammal afflicted with a cancer expressing TAA using immunocytochemistry. Such assessment of the delineation of the a TAA antigen in diseased tissue can be used to prognose the progression of the disease in a mammal afflicted with the disease or the efficacy of immunotherapy. In this embodiment, examples of TAAs include but are not limited to CEA, PSA, and MUC-1. Conventional methods for immunohistochemistry are described in (Harlow and Lane (eds) (1988) In “Antibodies A Laboratory Manual”, Cold Spinning Harbor Press, Cold Spring Harbor, N.Y.; Ausubel et al. (eds) (1987). In Current Protocols In Molecular Biology, John Wiley and Sons (New York, N.Y.).

In another embodiment the antibodies of the present invention are used for immunotherapy. The antibodies of the present invention may be used in passive immunotherapy.

In providing a patient with the antibodies or antigen binding fragments to a recipient mammal, preferably a human, the dosage of administered antibodies or antigen binding fragments will vary depending upon such factors as the mammal's age, weight, height, sex, general medical condition, previous medical condition and the like.

The antibodies or antigen-binding fragments of the present invention are intended to be provided to the recipient subject in an amount sufficient to prevent, lessen or attenuate the severity, extent or duration of the disease or infection.

Anti-idiotypic antibodies arise normally during the course of immune responses, and a portion of the anti-idiotype antibody resembles the epitope that induced the original immune response. In the present invention, the immunoglobulin gene or portion thereof of an antibody whose binding site reflects a target antigen of a disease state, is incorporated into the genome or portion thereof of a virus genome, alone or in combination with a gene or portion thereof of multiple immunostimulatory molecules, the resulting recombinant virus is able to elicit enhanced cellular and humoral immune response to the antigen used in combination with a recombinant replication-defective virus encoding GM-CSF.

The present invention provides for host cells infected with the recombinant replication-defective virus encoding GM-CSF and expressing the GM-CSF into the surrounding mileau. The host cells may also express one or more endogenous target antigens or immunological epitopes thereof or may be engineered to express one or more exogenous, foreign target antigens or immunological epitopes thereof which may be provided by a second recombinant vector. The recombinant vector encoding one or more target antigens or immunological epitopes thereof may also have foreign nucleic acid sequences encoding one or more costimulatory molecules and/or cytokines.

The host cells of the present invention included but are not limited to tumor cells, antigen presenting cells, such as PBMC, dendritic cells, cells of the skin or muscle, and the like. Antigen presenting cells include, but are not limited to, monocytes, macrophages, dendritic cells, progenitor dendritic cells, Langerhans cells, splenocytes, B-cells, tumor cells, muscle cells, epithelial cells and the like.

In one embodiment, the host cells are tumor cells in which the tumor cells are exposed to the recombinant replication-defective virus encoding GM-CSF in situ or in vitro to cause expression and secretion of GM-CSF by the tumor cells. The tumor cells may express an endogenous target antigen or the tumor cells may be further genetically engineered using a recombinant vector to express a target antigen such as TAA or immunological epitope thereof, and optionally to express one or more immunostimulatory molecules. Tumor cells expressing GM-CSF provided by the recombinant replication-defective virus along with an endogenous or exogenously provided TAA, and optionally expressing one with multiple immunostimulatory molecules are administered to a mammal in an effective amount to result in tumor reduction or elimination in the mammal afflicted with a cancer.

In one embodiment, the recombinant replication-defective virus encoding GM-CSF is directly injected into a tumor in situ such as in melanoma or metastatic breast cancer skin lesions. The recombinant replication-defective virus encoding GM-CSF may also be administered in situ during the time of surgery for cancers such as colorectal and pancreatic cancers. In addition to providing the recombinant replication-defective virus encoding GM-CSF, a vector encoding one or more immunostimulatory molecules may be provided for enhanced anti-tumor response. In one embodiment, the vector is a recombinant avipox encoding B7.1 or recombinant avipox encoding B7. l/LFA-3/ICAM-1. In another embodiment, the recombinant replication-defective virus encoding GM-CSF is provided in combination with a cytokine such as IL-12 or a vector encoding IL-12.

In another embodiment, the recombinant replication-defective virus encoding GM-CSF is provided by intra-lymph node injection. The lymph node site may be either distal to or draining a tumor site. The recombinant replication-defective virus encoding GM-CSF may be provided alone, or in combination with an target antigen or immunological epitope thereof, or a recombinant vector encoding a target antigen or immunological epitope thereof. The recombinant vector encoding a target antigen or immunological epitope thereof may further encode one or more immunostimulatory molecules. In one embodiment, the combination thereapy comprises recombinant replication-defective virus encoding GM-CSF and a recombinant vector encoding a target antigen or immunological epitope thereof and further encoding the costimulatory molecule B7.1. In another embodiment, a recombinant vector encoding a target antigen or immunological epitope thereof and further encoding B7.1/LFA-3/ICAM-1 is provided intranodally in combination with the recombinant replication-defective virus encoding GM-CSF.

Tumor cells may also be infected ex vivo using the recombinant replication-defective virus encoding GM-CSF, alone, or in combination with a recombinant vector encoding at least one or more immunostimulatory molecules for use as a vaccine. In one example, the recombinant vector is a recombinat avipox encoding B7.1. In another embodiment, the recombinant vector encodes B7.1/LFA-3/ICAM-1. In another example the recombinant vector encodes a cytokine such as gamma or alpha interferon The tumor cells may be from the same patient (autologous) or a cell line(s) from different patients (allogeneic). Administration of the tumor cells of the present invention provide an antitumor immune response to an individual. The tumor cells may be provided subcutaneously, intradermally, intravenously, and the like.

The present invention also provides progenitor dendritic cells, dendritic cells (DC), DC subpopulations, and derivatives thereof expressing GM-CSF in which the GM-CSF is exogenously provided by a recombinant replication-defective virus having nucleic acid sequences encoding GM-CSF. The APCs such as progenitor dendritic cells and dendritic cells may also express one or more endogenous target antigens or immunological epitopes thereof or exogenous target antigens may be provided by a recombinant vector. The recombinant vector may additionally encode one or more costimulatory molecules. In one embodiment, the dendritic cells are infected with a replication-defective virus encoding-GM-CSF and a recombinant vector encoding at least one target antigen. In another embodiment, the dendritic cells are infected with a replication-defective virus encoding-GM-CSF and with a recombinant avipox encoding at least one target antigen and encoding B7.1. In yet another embodiment, the dendritic cells are infected with a replication-defective virus encoding GM-CSF and a recombinant avipox encoding target antigen and encoding B7.1/LFA-3/ICAM-1. The present invention further provides methods of using the APCs, in activating T cells in vivo or in vitro for vaccination and immunotherapeutic responses against one or more target cells, target antigens and immunological epitopes thereof.

The APCs such as progenitor dendritic cells, dendritic cells, DC subpopulations and derivatives thereof isolated from a source infected with a recombinant replication-defective virus encoding GM-CSF, alone or in combination with a recombinant vector encoding B7 or B7/LFA-3/ICAM-1 may also be pulsed or incubated with at least one S peptide, protein, antibody, target cell, target cell lysate, cell extract, target cell membrane, apoptotic bodies, target antigen, or immunological epitope thereof, or with RNA or DNA of at least one target cell and administered to a species in an amount sufficient to activate the relevant T cell responses in vivo. In another embodiment, the antigen presenting progenitor dendritic cells and dendritic cells additionally express at least one foreign target antigen or immunological epitope thereof.

Host cells may be provided in a dose of 10³ to 10⁹ cells. Routes of administration that may be used include intravenous, subcutaneous, intralymphatic, intratumoral, intradermal, intramuscular, intraperitoneal, intrarectal, intravaginal, intranasal, oral, via bladder instillation, via scarification, and the like.

In one embodiment, the GM-CSF expressing antigen presenting progenitor dendritic cells or dendritic cells are exposed to a target cell, target cell lysates, target cell membranes, target antigen or immunological epitope thereof or with DNA or RNA from at least one target cell in vitro and incubated with primed or unprimed T cells to activate the relevant T cell responses in vitro. The activated T cells alone or in combination with the progenitor DC or DC are then administered to a species such as a human for vaccination or immunotherapy against a target cell, target antigen or immunological epitope thereof. In one method of use, the progenitor dendritic cells or dendritic cells are advantageously used to elicit an immunotherapeutic growth inhibiting response against cancer cells.

In another embodiment, the GM-CSF expressing antigen-presenting cell, preferably a precursor DC or DC is fused with a target cell expressing a relevant target antigen or immunological epitope thereof to form a heterokaryon of APC and target cell by methods known in the art (Gong, J. et al Proc. Natl. Acad. Sci. USA 95:6279-6283, 1998). Such a fusion cell or chimeric APC/target antigen cell expresses both GM-CSF and target antigen or immunological epitopes thereof. The APC may also be infected with a recombinant vector encoding at least one costimulatory molecule, preferably encoding B7.1 or B7.1/LFA-3/ICAM-1. In a preferred embodiment the target cell is a hyperplastic cell, premalignant or malignant cell. The chimeric APC/target antigen cell may be used both in vivo and in vitro to enhance immune responses of T and B lymphocytes.

Progenitor dendritic cells are obtained from bone marrow, peripheral blood and lymph nodes from a patient. The patient may have been previously vaccinated, or treated with a compound such as Flt-3L to enhance the number of antigen-presenting cells. Dendritic cells are obtained from any tissue such as the epidermis of the skin (Langerhans cells) and lymphoid tissues such as found in the spleen, bone marrow, lymph nodes, and thymus as well as the circulatory system including blood and lymph (veiled cells). Cord blood is another source of dendritic cells.

Dendritic cells may be enriched or isolated for use in the present invention using methods known in the art such as those described in U.S. Pat. No. 5,788,963. Once the progenitor dendritic cells, dendritic cells and derivatives thereof are obtained, they are cultured under appropriate culture conditions to expand the cell population and/or maintain the cells in a state for optimal infection, transfection or transduction by a recombinant vector and for optimal target antigen uptake, processing and presentation. Particularly advantageous for maintenance of the proper state of maturity of dendritic cells in in vitro culture is the presence of both the granulocyte/macrophage colony stimulating factor (GM-CSF) and interleukin 4 (IL-4). Subpopulations of dendritic cells may be isolated based in adherence and/or degree of maturity based on cell surface markers. The phenotype of the progenitor DC, DC and subpopulations thereof are disclosed in Banchereau and Steinman Nature 392:245-252, 1998.

In one embodiment GM-CSF and IL-4 are each provided in a concentration of about 500 units/ml for a period of about 6 days. In another embodiment, TNFα and/or CD40 is used to cause precursor DC or DC to mature.

The progenitor dendritic cells or dendritic cells may be obtained from the individual to be treated and as such are autologous in terms of relevant HLA antigens or the cells may be obtained from an individual whose relevant HLA antigens (both class I and II, e.g. HLA-A, B, C and DR) match the individual that is to be treated. Alternatively, the progenitor dendritic cell is engineered to express the appropriate, relevant HLA antigens of the individual receiving treatment.

The progenitor dendritic cells and dendritic cells may be further genetically modified to extend their lifespan by such methods as EBV-transformation as disclosed in U.S. Pat. No. 5,788,963.

The dendritic cells and precursors thereof may be provided in the form of a pharmaceutical composition in a physiologically acceptable medium. The composition may further comprise a target cell, target cell lysate, target cell membrane, target antigen or immunological epitope thereof. The composition may additionally comprise cytokines and/or chemokines such as IL-4 and GM-CSF for additional synergistic enhancement of an immune response.

Another aspect of the invention is the use of the recombinant replication-defective virus encoding GM-CSF for the prevention and treatment of neutropenia. Neutropenia is the medical term for an abnormally low number of neutrophils in the circulating blood. There are many potential causes of neutropenia which include: bone marrow damage from certain types of leukemias, lymphomas or metastatic cancers; an adverse reaction to a medication such as a diuretic or anti-depressant; response to radiation treatment or chemotherapy; the presence of an indwelling I.V. catheter; a viral infection such as infectious mononucleosis or HIV infection; a bacterial infection such as tuberculosis, an autoimmune disease such as systemic lupus erythematosus, congenital defects; impaired phagocytic, microbial and tumoricidal function of neutrophils, monocytes and macrophages; malnutrition; neoplastic obstruction of respiratory, digestive or urinary tracts complicated by secondary infections. Individuals with neutropenia get infections easily and often. Most of the infections occur in the lungs, mouth and throat (mucositosis), sinuses and skin. Painful mouth ulcers, gum infections, ear infections and peridontal disease are common. Severe life-threatening infections may occur requiring hospitalization and intravenous antibiotics.

The recombinant replication-defective virus encoding GM-CSF is useful in methods of preventing or treating neutropenia. The replication-defective virus encoding GM-CSF provides a quick and sustained concentration of GM-CSF, superior to administration of naturally-derived or recombinantly produce GM-CSF (Mangi, M. H. and Newland, A. C. 1999, European J. of Cancer. Vol. 35; Suppl. 3, pp. S4-S7).

The recombinant replication-defective virus encoding GM-CSF may be provided prior to (prophylactic) or after the development of neutropenia. A dose is administered in an amount effective to increase the numbers of neutrophils, preferably to increase the number of neutrophils to within a normal range. The dose may be provided one or more times.

The recombinant replication-defective virus encoding GM-CSF may be provided alone, or in combination with another therapy such as an antibiotic, antifungal, antiviral, and the like for treatment of infections. One or more antibiotics which may be included in a composition with the recombinant replication-defective virus encoding GM-CSF include but are not limited to ceftazidime, cefepime, imipenem, aminoglycoside, vancomycin, antipseudomonal β-lactam, and the like. One or more antifungal which may be included in a composition with the recombinant replication-defective virus encoding GM-CSF include but are not limited to amphotericin B, dapsone, fluconazole, flucytosine, griseofluvin, intraconazole, ketoconazole, miconazole, clotrimazole, nystatin, combinations thereof and the like. One or more antiviral agents may be included in a composition with the recombinant replication-defective virus encoding GM-CSF and include but are not limited to 2′-beta-fluoro-2′,3′-dideoxyadenosine, indinavir, nelfinavir, ritonavir, nevirapine, AZT, ddI, ddC, combinations thereof and the like.

In the case of irradiation treatment, chemotherapy or corticosteroid therapy which may result in neutropenia, the recombinant replication-defective virus encoding GM-CSF may be provided prior to the initiation of the irradiation, chemotherapy or corticosteroid therapy, concurrently with the therapy, or the recombinant replication-defective virus encoding GM-CSF may be provided after the irradiation, chemotherapeutic or corticosteriod treatment. The dose of the recombinant replication-defective virus encoding GM-CSF is provided in an amount to maintain normal numbers of neutrophils in the blood or to increase the number of neutrophils to prevent or inhibit neutropenia and its sequelae. The composition comprising the recombinant-replication defective virus encoding GM-CSF may also comprise a chemotherapeutic agent, a corticosteriod, or combinations thereof.

Another aspect of the invention is the use of the recombinant replication-defective virus encoding GM-CSF for the treatment of myeloidysplastic syndromes and cytopenias associated with myeloidysplastic syndromes in combination with erythropoietin (EPO) or preferably recombinant erythropoietin (rhEPO). Myelodysplastic syndromes (MDS) are a group of clonal stem cell disorders characterized by abnormal bone marrow differentiation and maturation, with quantitative as well as qualitative abnormalities within one or more haemopoietic cell lineages in the peripheral blood. The standard treatment for these individuals has been supportive care with blood products, antibiotics, and allogeneic bone marrow transplantation in selected younger individuals. Stasi, R et al reported the use of recombinant GM-CSF (rec GM-CSF) in combination with erythropoietin for treatment of cytopenias in patients with MDS (British J. Haematology 1999, 105, 141-148). However, rhGM-CSF is associated with significant side effects. In the present invention, recombinant replication-defective virus encoding GM-CSF is used in place of rec GM-CSF, in combination with EPO, for treatment of cytopenia associated with MDS. The recombinant replication-defective virus encoding GM-CSF is administered at a dose in the range of about 10⁵ to about 10 ¹⁰ pfu and provided once or at multiple intervals. The EPO is administered at a dose in the range of about 150-300 u/kg body weight and is provided at multiple intervals. The combined dose is effective in preventing or treating neutropenia, increase haemoglobin levels and/or reduce blood transfusion needs of the individual with MDS. The use of replication-defective virus encoding GM-CSF at weekly or monthly injections alleviates the need to administer recombinant GM-CSF protein daily.

GM-CSF has been shown to be useful as an adjuvant for immunotherapy with bispecific antibodies in cancer patients. (Elsasser, D. et al European J. Cancer, Vol. 35, Suppl. 3, pp. S25-S28, 1999). In the present invention, recombinant replication-defective virus encoding GM-CSF replaces GM-CSF for a superior adjuvant effect in combination with a bispecific antibody alleviating the need to administer recombinant GM-CSF protein daily. Bispecific antibodies are chemically or genetically-constructed molecules that combine specificity for the tumor cell antigen/epitope with reactivity for cytotoxic trigger molecules found on immune effector cells. The recombinant replication-defective virus encoding GM-CSF is provided in a dose range of about 10⁵ to about 10¹⁰ pfu at multiple intervals. A bispecific antibody is provided in a dose of about 0.2-200 mg/m² at multiple intervals such as weekly, monthly and the like. The criteria for enhanced immunotherpeutic response includes specific lytic activity, specific cytokine production, antibody-mediated cellular cytotoxicity, tumor regression, protection from tumor. Bispecific antibodies which may be used in combination with the recombinant replication-defective virus encoding GM-CSF include but are not limited to FcγRI (CD64), FcγRII (CD32), FcγRIII (CD16), anti-CD3-directed bispecific antibodies with tumor-directed specificities for HER-2/neu, EGF-receptor, CD15 antigen or the EpCAM molecule. (McCall, A. M., Adams, G. P., Amoroso, A. R., Nielsen, U. B., Zhang, L., Horak, E., Simmons, H., Schler, R., Marks, J. D. and Weinder, L. M. Isolation and characterization of an anti-CD16 single-chain Fv fragment and construction of an anti-HER2/neu/anti-CD16 bispecific scFv that triggers CD16-dependent tumor cytolysis. Mol. Immunol. 36:433-445, 1999).

The description of the specific embodiments will so fully reveal the general nature of the invention that others can readily modify and/or adopt for various purposes such specific embodiments without departing from the generic concept, and therefor such adaptations and modifications are intended to be comprehended within the meaning and range of equivalents of the disclosed embodiments.

All references and patents referred to are incorporated herein by reference.

### EXAMPLE 1

### Generation of Recombinant Viruses

The generation of recombinant poxviruses is accomplished via homologous recombination in vivo between poxvirus genomic DNA and a plasmid vector that carries the heterologous sequences to be inserted. Plasmid vectors for the insertion of foreign sequences into poxviuses are constructed by standard methods of recombinant DNA technology (Sambrook et al 1989). The plasmid vectors contain one or more chimeric genes, each comprising a poxvirus promoter linked to a protein coding sequence, flanked by viral sequences from a non-essential region of the poxvirus genome. The plasmid is transfected into cells infected with the parental poxvirus, and recombination between poxvirus sequences on the plasmid and the corresponding DNA in the viral genome results in the insertion into the viral genome of the chimeric genes on the plasmid Recombinant viruses are selected and purified using any of a variety of selection or screening systems (Mazzara et al, 1993; Jenkins et al, 1991; Sutter et al, 1994), several of which are described below. Insertion of the foreign genes into the vaccinia genome is confirmed by polymerase chain reaction (PCR) analysis. Expression of the foreign genes is demonstrated by Western analysis.

### Origin of Fowlpox Parental Virus

The parental fowlpox virus used for the generation of recombinants was plaque-purified from a vial of USDA-licensed poultry vaccine, POXVAC-TC, which is manufactured by Schering-Plough Corporation. The starting material for the production of POXVAC-TC was a vial of Vineland Laboratories' chicken embryo origin Fowl Pox vaccine, obtained by Schering-Plough. The virus was passaged twice on the chorioallantoic membrane of chicken eggs to produce a master seed virus. The master seed virus was passaged 27 additional times in chicken embryo fibroblasts to prepare the POXVAC-TC master seed. To prepare virus stocks for the generation of POXVAC-TC product lots, the POXVAC-TC master seed was passaged twice on chicken embryo fibroblasts. One vial of POXVAC-TC, serial #96125, was plaque-purified three times on primary chick embryo dermal cells.

### Origin of Vaccinia Parental Virus

The virus is the New York City Board of Health strain and was obtained by Wyeth from the New York City Board of Health and passaged in calves to create the Smallpox Vaccine Seed. Flow Laboratories received a lyophilized vial of the Smallpox Vaccine Seed, Lot 3197, Passage 28 from Drs. Chanock and Moss (National Institutes of Health). This seed virus was ether-treated and plaque-purified three times.

### Origin of Modified Vaccinia Virus Ankara (MVA) Parental Virus

MVA was derived from the Ankara vaccinia strain CVA (Mayr et at, 1975). Virus attenuation was carried out by terminal dilution in chick embryo fibroblasts (CEFs). After 360 passages, the virus was plaque-purified three times and then further passaged in CEFs. At passage 516, the attenuated CVA virus was renamed MVA. After 570 passages, the virus was again plaque-purified and further passaged. Seed virus passage 575 was obtained from Dr. Anton Mayr and was plaque-purified twice on primary chick embryo dermal cells.

### Generation of Recombinant Poxviruses

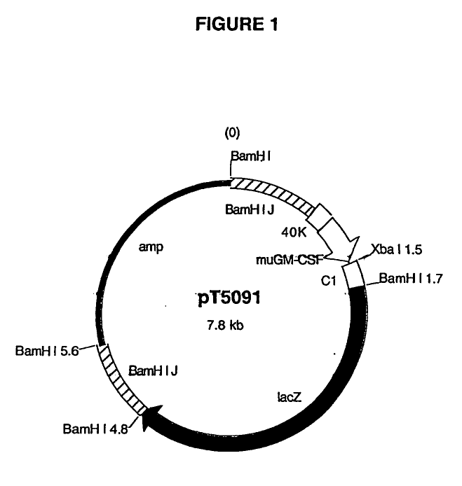

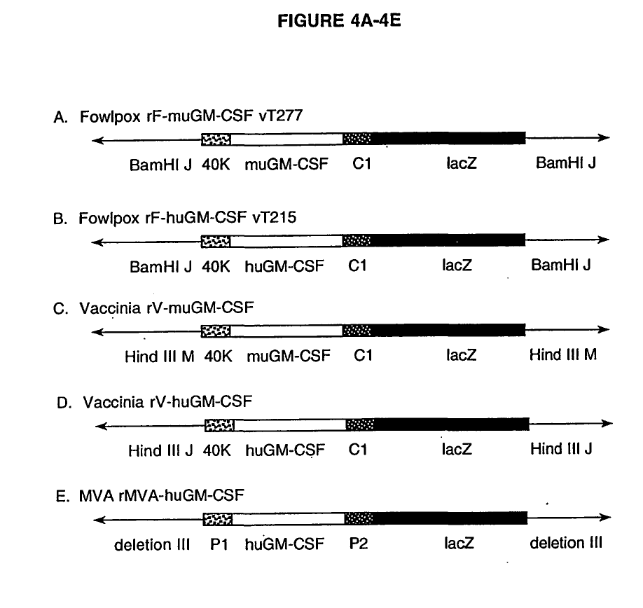

For the generation of rF-muGM-CSF, a plasmid vector, designated pT5091 (FIG. 1), was constructed to direct insertion of the foreign sequences into the BamHI J region of the fowlpox genome. The murine GM-CSF gene is under the control of the vaccinia 40K promoter (Gritz et al, 1990). In addition, the E. coli lacZ gene, under the control of the fowlpox virus C1 promoter (Jenkins et al, 1991), is included as a screen for recombinant progeny. These foreign sequences are flanked by DNA sequences from the BamHI J region of the fowlpox genome. A plaque-purified isolate from the POXVAC-TC strain of fowlpox was used as the parental virus for this recombinant vaccine. The generation of recombinant fowlpox virus was accomplished via homologous recombination between fowlpox sequences in the fowlpox genome and the corresponding sequences in pT5091 in fowlpox-infected primary chick embryo dermal cells transfected with pT5091. Recombinant virus was identified using a chromogenic assay, performed on viral plaques in situ, that detects expression of the lacZ gene product in the presence of halogenated indolyl-beta-D-galactoside (Bluo-gal), as described previously (Chakrabarti et al, 1985). Viral plaques expressing lacZ appear blue against a clear background. Positive plaques, designated vT277 (FIG. 4A), were picked from the cell monolayer and their progeny were replated. Four rounds of plaque isolation and replating in the presence of Bluo-Gal resulted in the purification of the desired recombinant.

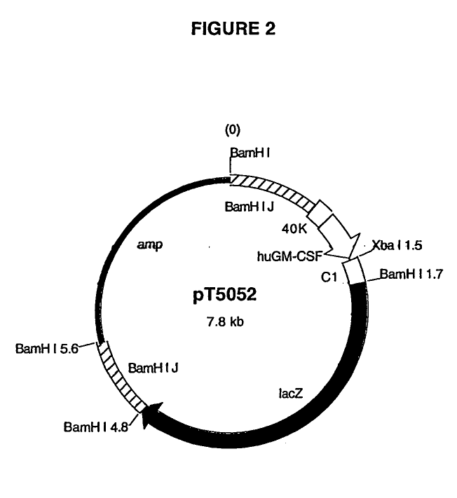

For the generation of rF-huGM-CSF, a plasmid vector, designated pT5052 (FIG. 2), was constructed to direct insertion of the foreign sequences into the BamHI J region of the fowlpox genome. Plasmid vector pT5052 was deposited with the American Type Culture Collection, 10801 University Boulevard, Manassas, Va. 20110 under the terms of the Budapest Treaty on Jun. 15, 2000 under Accession No. PTA-2099. The human GM-CSF gene is under the control of the vaccinia 40K promoter and the lacZ gene is under the control of the C1 promoter. These foreign sequences are flanked by DNA sequences from the BamHI J region of the fowlpox genome. A plaque-purified isolate from the POXVAC-TC (Schering-Plough Corporation) strain of fowlpox was used as the parental virus for this recombinant vaccine. The generation of recombinant vaccinia virus was accomplished via homologous recombination between fowlpox sequences in the fowlpox genome and the corresponding sequences in pT5052 in fowlpox-infected primary chick embryo dermal cells transfected with pT5052. Recombinant virus was identified using the chromogenic assay for the lacZ gene product described above. Viral plaques expressing lacZ appear blue against a clear background. Positive plaques, designated vT215 (FIG. 4B), were picked from the cell monolayer and their progeny were replated. Five rounds of plaque isolation and replating in the presence of Bluo-Gal resulted in the purification of the desired recombinant.

For the generation of a recombinant fowlpox virus that co-expresses a tumor-associated antigen (TAA) and GM-CSF, designed rF-TAA/GM-CSF, a plasmid vector is constructed to direct insertion of the foreign sequences into the fowlpox virus genome. The TAA gene and GM-CSF gene are under the control of a multiplicity of promoters. These foreign sequences are flanked by DNA sequences from the fowlpox virus genome into which the foreign sequences are to be inserted. The generation of recombinant fowlpox virus is accomplished via homologous recombination between fowlpox virus sequences in the fowlpox virus genome and the corresponding sequences in the plasmid vector in fowlpox virus-infected cells transfected with the plasmid vector. Recombinant plaques are picked from the cell monolayer under selective conditions, as described above, and their below. Oligonucleotides A through E, which overlap the translation initiation codon of the H6 promoter with the ATG of rabies G, were cloned into pUC9 as pRW737. Oligonucleotides A through E contain the H6 promoter, starting at NruI, through the HindIII site of rabies G followed by BgIII. Sequences of oligonucleotides A through E ((SEQ ID NO:42)-(SEQ ID NO:46)) are:

| | | |
|---|---|---|
| A: | CTGAAATTATTTCATTATCGCGATATCCGTTA | (SEQ ID NO:42) |
| | AGTTTGTATCGTAATGGTTCCTCAGGCTCTCC | |
| | TGTTTGT | |
| B: | CATTACGATACAAACTTAACGGATATCGCGAT | (SEQ ID NO:43) |
| | AATGAAATAATTTCAG | |
| C: | ACCCCTTCTGGTTTTTCCGTTGTGTTTTGGGA | (SEQ ID NO:44) |
| | AATTCCCTATTTACACGATCCCAGACAAGCTT | |
| | AGATCTCAG | |
| D: | CTGAGATCTAAGCTTGTCTGGGATCGTGTAAA | (SEQ ID NO:45) |
| | TAGGGAATTTCCCAAAACA | |
| E: | CAACGGAAAAACCAGAAGGGGTACAAACAGGA | (SEQ ID NO:46) |
| | GAGCCTGAGGAAC | |

The diagram of annealed oligonucleotides A through E is as follows:

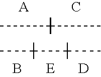

Oligonucleotides A through E were kinased, annealed (95° C. for 5 minutes, then cooled to room temperature), and inserted between the PvuII sites of pUC9. The resulting plasmid, pRW737, was cut with HindIII and BgIII and used as a vector for the 1.6 kbp HindIII-BgIII fragment of ptg155PRO (Kieny et al., 1984) generating pRW739. The ptg155PRO HindIII site is 86 bp downstream of the rabies G translation initiation codon. BgIII is downstream of the rabies G translation stop codon in ptg155PRO. pRW739 was partially cut with NruI, completely cut with BgIII, and a 1.7 kbp NruI-BgIII fragment, containing the 3′ end of the H6 promoter previously described (Taylor et al., 1988a,b; Guo et al., picked from the cell monolayer under selective conditions and their progeny are further propagated. Additional rounds of plaque isolation and replating result in the purification of the desired recombinant virus (FIG. 4D).

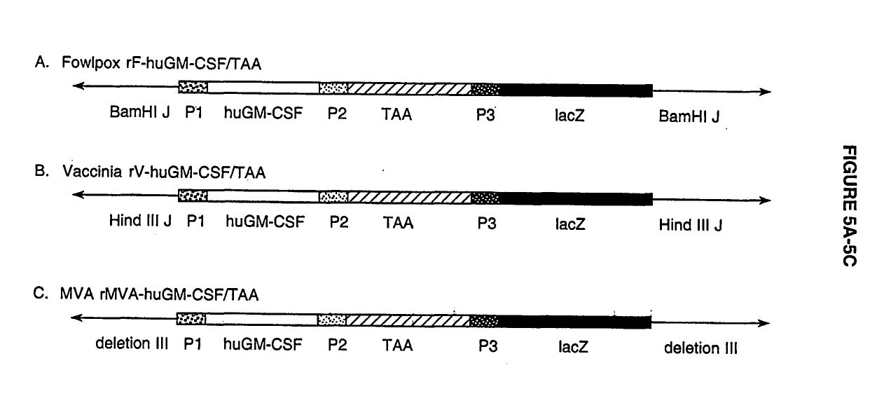

For the generation of a recombinant vaccinia virus that co-expresses a tumor-associated antigen (TAA) and GM-CSF, designated rV-TAA/GM-CSF, a plasmid vector is constructed to direct insertion of the foreign sequences into the vaccinia genome. The TAA gene and the GM-CSF gene are under the control of a poxvirus promoter. These foreign sequences are flanked by DNA sequences from the vaccinia genome into which the foreign sequences are to be inserted. The generaton of recombinant vaccinia virus is accomplished via homologous recombination between vaccinia sequences in the vaccinia genome and the corresponding sequences in the plasmid vector in vaccinia-infected cell transfected with the plasmid vector. Recombinant plaques are picked from the cell monolayer under selective conditions, as described above, and their progeny are further propagated. Additional rounds of plaque isolation and replating result in the purification of the desired recombinant virus (FIG. 5B).

For the generation of a recombinant MVA that expresses GM-CSF, a plasmid vector is constructed to direct insertion of the foreign sequences into the MVA genome. The GM-CSF gene is under the control of a poxviral promoter. These foreign sequences are flanked by DNA sequences from the MVA genome into which the foreign sequences are to be inserted, for example, deletion III (Sutter et al, 1994). The generation of recombinant MVA is accomplished via homologous recombination between MVA sequences in the MVA genome and the corresponding sequences in the plasmid vector in MVA-infected cells transfected with the plasmid vector. Recombinant plaques are picked from the cell monoloayer under selective conditions and their progeny are further propagated. Additional rounds of plaque isolation and replating result in the purification of the desired recombinant virus (FIG. 4E). The genomic structure of a recombinant MVA coexpressing GM-CSF with a tumor-associated antigen (TAA) is shown in FIG. 5C.

### EXAMPLE 2

### Materials and Methods

Animals, cell lines and reagents. CEA.Tg mice (H-2^{b}) (line 2682) were provided by Dr. John Thompson, Institute of Immunobiology, University of Freiburg, Freiburg, Germany (24). A cosmid clone containing the complete coding region of the human CEA gene, including 3.3 kb of the 5′-flanking region and 5 kb of the 3′-flanking region, was used to generate the CEA. Tg mice (30). CEA protein expresison was found predominately in the gastrointestinal tract, whereas other sites, such the trachea, esophagus, small intestine, and lung, also expressed CEA. The mice were housed and maintained in microisolator cages under specific pathogen-free conditions. Lines were established from founder animals by continuous backcrossing with wild-type, female B6 mice. CEA-positive offspring were identified by the presence of fecal CEA detected using a solid-phase, double-determinant anti-CEA ELISA kit (AMDL, Inc. Tustin, Calif.).

The CEA-expressing MC-38 cells, designated MC-38-CEA-2 (H-2^{b}), were produced by transducing the human CEA gene using the retroviral expression vector pBNC (32). The line was cloned and routinely examined by flow cytometry for stable CEA expression as measured by COL-1 (33) reactivity. Both the parental MC-38 and MC-38-CEA-2 cell lines were grown in DMEM containing high glucose and 10% heat-inactivated FBS. FDCP-1 cells were kindly provided by Dr. Jim lhle (St. Jude's Hospital, Memphis, Tenn.) and grown in RPMI 1640 supplemented with 2 mM L-glutamine, 50 μM 2-mercaptoethanol, 10% heat-inactivated FBS, 50 μg/ml gentamicin and 10% WEHI cell culture supernatant. Lyophilized recombinant murine GM-CSF was obtained from PeproTech, Inc. (Rock Hill, N.J.) and stored at −80° C. until use. Prior to use, recGM-CSF was reconstituted to the appropriate concentration with saline containing 1% mouse serum. Reconstituted recGM-CSF was also stored at −20° C. and its biological activity was checked every 3-6 months using the GM-CSF-dependent FDCP-1 indicator cells (34).

Recombinant Avian Poxviruses. The recombinant avian poxviruses used in the study were fowlpox and canarypox (ALVAC) virus-based vectors. To simplify the narrative, they are collectively referred to as recombinant avipox viruses. The individual recombinant avipox viruses used to generate the data presented in each Table and Figure are identified as avipox(F)- and avipox(A) for the fowlpox and canarypox (ALVAC) vectors, respectively.

Avipox(F)-GM-CSF. The parental virus used for the generation of rF-GM-CSF (i.e., avipox(F)-GM-CSF) was plaque-purified from a tissue-culture adapted vaccine strain of fowlpox virus. Avipox(F)-GM-CSF was constructed via homologous recombination in vivo between the parental fowlpox DNA and a plasmid vector that contains the murine GM-CSF gene. The recombinant virus, produced at Therion Biologics Corp. (Cambridge, Mass.), was then used to generate a seed stock, which was characterized by genomic and protein expression analysis.

Avipox(A)-recombinants. Avipox(A) is a canarypox virus-based vector that is restricted to avian species for productive replication (35). The canary pox strain was isolated from a pox lesion on an infected canary and attenuated by 200 serial passages in chick embryo fibroblasts and was subjected to four successive rounds of plaque purification under agarose. All amplifications and plaque titrations were performed on primary chick embryo fibroblasts. Avipox(A)-GM-CSF (vCP319), avipox(A)-rabies glycoprotein G (designated avipox(A)-RG, vCP65) and avipox(A)-CEA (vCP248) were kindly supplied by Virogenetics Corp (Troy, N.Y.). GM-CSF expression was confirmed by a bioassay (see below) and CEA expression by Western blot analysis using the murine monoclonal antibody COL-1 (32).

In Vitro GM-CSF Production. MC-38 cells were trypsinized and washed twice in serum-free Opti-MEM (Life Technologies Co., Gaithersburg, Md.). Four million cells were placed in 15 ml conical polypropylene tubes and pelleted by centrifugation. The cell pellet was resuspended in 300 μl Opti-MEM to which 10 μl of either avipox-GM-CSF or appropriate control viruses at the indicated pfu were added. Infected cells were incubated at 37° C. for 1 h and agitated every 10-15 min. Following incubation, the cells were washed 2× in 10 ml growth medium supplemented with 10% FBS. Viable cells were counted using trypan blue exclusion, and 3×10⁵ cells were added per well in 6-well plates. Supernatants were harvested 24, 48 and 72 h later, and the level of biologically active GM-CSF was determined as outlined above.

Regional Lymph Node Analyses. Female C57BL/6 (B6) mice (H-2^{b}) were obtained from the National Cancer Institute, Frederick Cancer Research and Development Facility (Frederick, Md.). Six- to eight-week-old mice were housed and maintained in microisolator cages under pathogen-free conditions. Recombinant avipox-GM-CSF viruses, appropriate control viruses (i.e., F-WT, avipox-RG) and recombinant GM-CSF protein were administered by s.c. injections at the base of the tail. Subiliac, para-aortic and sacral lymph nodes were surgically isolated, cells mechanically dispersed and transferred to a 50 ml conical tube. They were allowed to stand on ice for 10 minutes after which the supernatant was removed. The cells were pelleted by centrifugation (500×g) and washed twice in cold Ca²⁺—Mg²⁺-free DPBS. After the second wash, the cells were resuspended in Ca²⁺—Mg²⁺free DPBS at a concentration of 0.5-1.0×10⁶ cells/ml. They were aliquoted and approximately 10⁶ cells were incubated with 1 μg FiTc-labeled anti-I-A^{b} (BALB/c mouse, IgG2a,-k) or appropriate control antibody (PharMingen, Inc., San Diego, Calif.) for 1 h at 4° C. Samples also contained 1 μg of the unlabeled 2.2G2 antibody (CD16) to block Fc receptors. After incubation, the cells were washed twice and immediately analyzed using a Becton-Dickinson FACScan equipped with a blue laser with an excitation of 15 mW at 488 nm. Data were gathered from 10,000 cells using a live gate, stored, and used for analysis.

Isolation of CD11c⁺ Cells. Regional lymph nodes, consisting of the subiliac, para-aortic and sacral nodes, were surgically removed and pooled from groups of untreated and treated mice and placed in RPMI-1640 containing 15 mM HEPES (pH 7.4 and 10% heat inactivated FBS. Cells were mechanically dispersed through a 70-μm cell stainer, transferred to a 50 ml conical tube and placed on ice. The cell suspensions were washed twice by centrifugation (500×g) in cold DPBS and incubated at 4° C. for 1 h in cold DPBS containing 1.5 ml/10⁸ cells of biotin-anti-CD11 c (clone B-ly6, PharMingen, Inc., San Diego, Calif.). Cells were centrifuged, washed 2× in DPBS and resuspended in 100 μl of MACS colloidal supra-paramagnetic MicroBeads conjugated to streptavidin (Miltenyi Biotec, Inc., Gladbach, Germany) and incubated at 4° C. for 15 min. The cells were washed with cold DPBS, pelleted by centrifugation (200×g) for 10 minutes and resuspended in 500 μl DPBS. A MACS LS⁺ separation column was placed within the MIDI MACS magnetic separator and prepared according to the manufacturer's instructions. The cell suspension was immediately applied onto the column and the non-magnetic cells were allowed to pass through The column was rinsed 3× with 3 ml buffer and removed from the magnetic separator, and the MACS⁺ cell fraction was eluted from the column. The MACS⁺ cell fraction was enriched with another application to the column and the number of cells in the MACS⁺ fraction was counted using a hemocytometer and by FACS. Flow cytometric analysis using a double stain consisting of a biotin-PE conjugate and an anti-A^{b}-FiTc antibodies (clone M5/114.15.2, IgG2b), revealed >80% of the MACS+ cell fraction were CD11 c⁺/I-Ab⁺, CD19⁻ and CD3⁻.

Mixed Lymphocyte Culture. For the mixed lymphocyte reaction, purified splenic BALB/c (H-2^{d} ) T cells were grown in an RPMI-1640 medium containing 10% heat-inactivated FIBS in the presence of irradiated C57BL/6 (H-2⁶) lymph node cells (1:1 ratio). After incubation for 5 days @ 37° C. in T-25 flasks, viable T cells were recovered from culture by density centrifugation over a Ficoll-Hypaque gradient and used in a unidirectional CTL assay with MC-38 (H-2^{b}) and P815 (H-2^{d}) serving as targets.

Immunizations. CEA.Tg mice were immunized by s.c. injection of avipox-CEA or avipox-RG in 100 μl near the base of the tail. Where indicated, recombinant avipox-GM-CSF viruses or recGM-CSF were mixed with avipox-CEA prior to injection. Recombinant GM-CSF protein was subsequently administered to mice daily for 3-4 consecutive days at the immunization site.

Serum Antibody Responses. Serum samples were collected from wild-type B6 as well as CEA.Tg and analyzed for the presence of antibodies to the appropriate target antigen by ELISA-Microtiter plates were sensitized overnight at 4° C. with 100 ng/well CEA (International Enzymes, Fallbrook, Calif.), OVA (Sigma Chemicals), murine recGM-CSF or 5×10⁵ pfu/well ALVAC. Wells were blocked with PBS containing 5% BSA, followed by a 1 h incubation of diluted mouse serum (1:10 to 1:31,250). After incubation, excess liquid was aspirated and plates were washed 3-5× with buffer (PBS containing 1% BSA). Antibodies bound to the wells were detected with HRP-conjugated goat anti-mouse IgG (Kirkegaard & Perry Labs., Inc., Gaithersburg, Md.) or IgM (Jackson lmmunoResearch, West Grove, Pa.). After a 1 h incubation, the level of reactivity was detected with the addition of chromogen, o-phenylenediamine, for 10 min and read using an ELISA microplate autoreader EL310 (Bio-Tek Instruments, Inc., Winooski, Vt.) at A_{490 nm}. Triplicates of positive and negative controls and serum samples were run for all assays. Positive controls for CEA and ALVAC were a murine IgG2a anti-CEA MAb, COL-1 (35), and a polyclonal rabbit anti-ALVAC IgG, respectively, which were developed in the laboratory. A commercially available rat anti-mouse GM-CSF monoclonal antibody (clone MP1-22E9, PharMingen, Inc., San Diego, Calif.) was used as a positive control in the anti-GM-CSF ELISA assays. Antibody titers were determined as the reciprocal of the serum dilution that results in an A_{490 mn} of 0.5.

T-cell Proliferation Assay. Mouse splenocytes were enriched for T cells by magnetic murine pan B (B220) Dynabeads (Dynal, A. S., Oslo, Norway), and FACS analysis showed that the resulting cell population was >95% CD3⁺. The isolated T lymphocytes were resuspended in RPMI 1640 containing 15 mM HEPES (pH 7.4), 10% heat-inactivated FBS, 2 mM L-glutamine, 0.1 mM non-essential amino acids, 1 mM sodium pyruvate, 50 u/ml gentamicin, and 50 μM β-mercaptoethanol. The assay consisted of coincubating 5×10⁵ irradiated splenocytes from noninmune, syngeneic B6 mice (serving as APC) and 1.5×10 purified splenic T lymphocytes in the presence of 50 μg/ml either CEA (Vitro Diagnostics, Littleton, Colo.), OVA or medium in each well of flat-bottom, 96-well plates. After 5 days in culture, the cells were pulsed with [3H]-thymidine (1 μCi/well; Amersham Corp., Arlington Heights, Ill.) and harvested 24 hr later, and the incorporated radioactivity was measured by liquid scintillation spectroscopy (Wallac, Inc., Gaithersburg, Md.).

CTL Lines and Cytotoxicity Assay. Four weeks after the second immunization with avipox-CEA/RG+avipox-GM-CSF or recGM-CSF, spleens from 2-3 mice/group were pooled and single cell suspensions were generated. Splenocytes were suspended in RPMI 1640 supplemented with 15 mM HEPES (pH 7.4),. 2 mM L-glutamine, 0.1 mM nonessential amino acids, 1 mM sodium pyruvate, 10 mg/ml gentamicin, 10% heat-inactivated FBS (Hyclone Laboratories, Logan, Utah) and 50 μM 2-ME. Twenty-five million splenocytes were added in 10 ml to T-25 flasks along with 10 μg/ml of a CEA ₅₂₆₋₅₃₃ (EAQNTTYL). T cell cultures were stimulated twice at weekly intervals by harvesting the T cells over a Ficoll-Hypaque gradient to remove dead cells and erythrocytes and incubating 2×10⁵ T-cells in the presence of 5×10⁶ irradiated syngeneic splenocytes, 10 μg CEA₅₂₆₋₅₃₃/ml and 10 U/ml recombinant human IL-2 (Proleukin, Chiron Corp., Emeryville, Calif.). Cytolytic activity was assessed following 2 in vitro stimulations using EL-4, a murine lymphoma cell line, pulsed with either CEA₅₂₆₋₅₃₃ or Flu NP 366-374.

CTL activity was assessed by using a modification of a previously described method (36). Overnight indium-111 (¹¹¹In) release assays were performed. Briefly, 4×10⁶ EL-4 target cells were radiolabeled with 50 μCi in (¹¹¹In)-Oxyquinoline (Amersham, Chicago, Ill.) for 30 min at 37° C. Peptide-pulsed target cells were incubated with 1 μg peptide/ml following labeling. Target and effector cells were mixed at the appropriate ratios and incubated for 18 hr at 37° C. The amount of ¹¹¹In released was measured in a gamma counter (Cobra Autogamma, Packard Instruments, Downers Grove, Ill.) and the percentage of specific lysis was calculated as follows:

% specific lysis=[(experimental cpm−spontaneous cpm)/(maximal cpm−spontaneous cpm)]×100. Lytic units (LU₃₀) indicate the number of effector cells required to obtain 30% lysis of 10,000 peptide-pulsed EL-4 cells.

Cytokine Production Assays. The T cell lines were incubated in flat-bottomed, 96-well plates at a cell density of 2×10⁴ cells/well, 5×10⁵ irradiated (2000 rad) syngeneic CEA.Tg mouse splenocytes/well and different concentrations of CEA 526-533 peptide. Supernatants were harvested after 48 h, and IFN-γ and IL-4 levels were measured using the appropriate ELISA assay (Endogen, Inc., Cambridge, Mass.).

Tumor Therapy Studies. Six- to eight-week-old male and female CEA.Tg mice were initially given a single i.p. injection of 2 mg cyclophosphamide. Four days later, 3×10⁵ cells MC-38-CEA-2 tumor cells (in 100 μl were injected s.c. in the right flank. FACS analysis of the injected cells showed CEA expression (COL-1 binding) on >85% of the cells, strong MHC class I, and the absence of MHC class II (I-A^{b}) expression. Four-5 days later, when the tumor volumes were 30-50 mm³, mice received the primary immunization of 10⁸ pfu avipox-CEA/RG alone or in combination with 10⁸ pfu avipox-GM-CSF or 20 μg recGM-CSF in 200 μl which was divided into 100 μl and injected s.c. on either side of the tail. RecGM-CSF was administered at the immunization site daily for 4 consecutive days. The immunization was boosted two weeks later at the same site. Tumors were measured 2-3×/week and the volumes calculated as: [(mm, short axis)² X (mm, long axis)]/2. Mice bearing tumors >2 CM³ were sacrificed for humane reasons and the day of death recorded. In those mice in which tumor volumes decrease, presumably due to immunization, the data were divided into two categories: (i) tumor regression and (ii) tumor eradication which were defined as a measured decrease in tumor volume and the complete disappearance of tumor, respectively. Mice in which the tumors were completely eradicated were challenged with a second s.c. injection of 3×10⁵ cells MC-38-CEA-2 tumor cells (in 100 μl) in the opposite flank.

Statistical Analysis. Statistical significance of T-cell proliferation/lysis data were based on Student's two-tailed t test. Differences in the growth rate of the MC-38-CEA-2 tumors as measured by changes in tumor volume for each treatment group were compared using the Mann-Whitney U test. In Table 3, tumor growth in individual CEA.Tg mice was divided into two categories: (i) tumor regression, defined by a measured reduction in tumor volume and (ii) tumor eradication, defined as the inability to measure or palpate tumor at the site of injection. All p values reported are two-sided and have not been adjusted for the multiplicity of evaluation performed on the data. A p value of <0.05 was considered significant.

### EXAMPLE 3

### GM-CSF Production by Recombinant Avipox Viruses

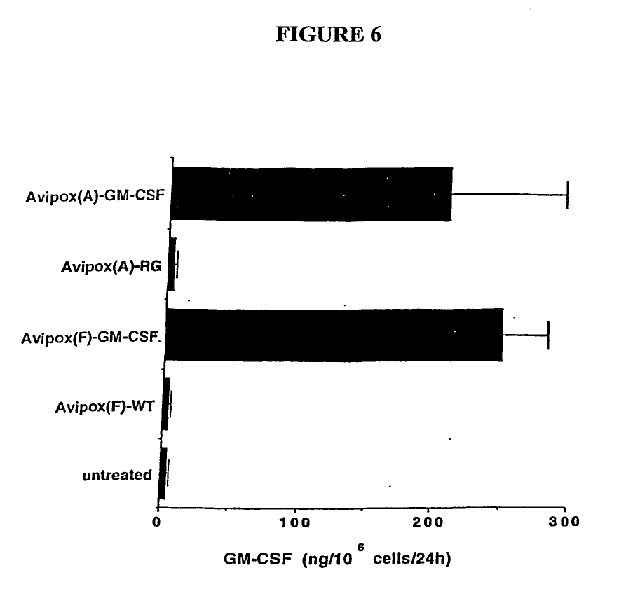

Recombinant avipox viruses expressing murine GM-CSF were generated and their ability to produce GM-CSF in vitro was assessed following infection of MC-38 tumor cells (FIG. 6). The recombinant avipox-GM-CSF viruses produced approximately equivalent amounts of GM-CSF (i.e., 225-250 ng/10⁶ cells/day) as determined in a bioassay using a GM-CSF-dependent cell line. Infection of the same cells with the same MOI of the control viruses produced no detectable GM-CSF.

### EXAMPLE 4

### Cellular/Functional Changes in Regional Lymph Nodes Following Avipox-GM-CSF or recGM-CSF Administration.

Enrichment of the draining regional lymph nodes with class II-expressing cells has been an in vivo readout for GM-CSF bioactivity in murine models (15, 22). Indeed, regional lymphadenopathy was observed in B6 mice seven days following the injection of 10⁷ or 10⁸ pfu of the recombinant avipox-GM-CSF viruses and to a lesser extent by the appropriate control viruses (Table 3).

TABLE 3
Cellular changes in regional nodes of B6 mice following the administration of avipox-GM-CSF or rGM-CSF^{a}.

| | | | Lymph Node Cellularity and Class II expression^{b} | | | | | |
|---|---|---|---|---|---|---|---|---|
| | | | DAY 7 | | | DAY 21 | | |
| | | | Cells/node | | | Cells/node | | |
| Treatment | Dose | # inj. | (×10 − 6) | % I-Ab⁺ cells | MFI | (×10 − 6) | % I-Ab⁺ cells | MFI |
| HBSS | N/A | N/A | 2.1 ± 0.1 | 25.4 ± 1.1 | 320-377 | 2.4 ± 0.1 | 28.9 ± 1.5 | 332-368 |
| avipox(F)-GM-CSF | 10⁷ pfu | 1 | 6.0 ± 0.4^{b,c} | 44.5 ± 6.2^{b,c} | 881-998 | 7.5 ± 0.8^{b,c} | 36.3 ± 2.2^{b,c} | 586-797 |
| | 10⁸ pfu | 1 | 24.4 ± 3.3^{b,c} | 43.4 ± 5.5^{b,c} | 1159-1778 | 8.1 ± 1.3^{b} | 41.9 ± 3.0^{b,c} | 840-1104 |
| avipox(F)-WT | 10⁷ pfu | 1 | 4.1 ± 0.8 | 25.9 ± 3.0 | 350-420 | 3.6 ± 0.2 | 23.8 ± 2.2 | 366-422 |
| | 10⁸ pfu | 1 | 14.4 ± 1.1^{b} | 24.5 ± 1.9 | 466-588 | 7.5 ± 3.3^{b} | 22.6 ± 1.2 | 366-515 |
| avipox(A)-GM-CSF | 10⁷ pfu | 1 | 7.2 ± 0.5^{b,c} | 53.2 ± 6.0^{b,c} | 661-814 | 6.2 ± 0.4^{b,c} | 4.80 ± 4.1^{b,c} | 560-620 |
| | 10⁸ pfu | 1 | 18.8 ± 1.1^{b,c} | 51.5 ± 4.8^{b,c} | 980-1228 | 9.5 ± 1.1^{b,c} | 45.5 ± 3.3^{b,c} | 888-1060 |
| avipox(A)-RG | 10⁷ pfu | 1 | 2.7 ± 0.3 | 28.0 ± 0.7 | 345-386 | 2.6 ± 0.2 | 29.2 ± 2.0 | 333-399 |
| | 10⁸ pfu | 1 | 9.5 ± 0.5^{b} | 24.1 ± 3.3 | 359-422 | 4.2 ± 0.2^{b} | 22.9 ± 0.8 | 345-410 |
| rGM-CSF | 20 μg | 1 | 3.1 ± 0.2^{b} | 25.9 ± 1.8 | 319-455 | 2.5 ± 0.1 | 28.1 ± 1.3 | 303-344 |
| | 20 μg | 4 | 7.1 ± 0.4^{b,d} | 39.8 ± 2.1^{b,d} | 844-1020 | 2.3 ± 0.2 | 26.1 ± 0.8 | 344-398 |
| ^{a}B6 mice (6-10 mice/group) were injected with the indicated recombinant avipox virus or with rGM-CSF as outlined in the Materials and Methods. Control mice received HBSS. Lymph nodes were removed on days 7 and 21 from the avipox-treated mice, and 24 h after the final injection from the rGM-CSF-treated mice. Total number of lymph node cells and class II expression levels were determined. Data are the mean ± SEM from two separate | | | | | | | | |
| # experiments. MFI values are expressed as a range of 4-6 determinations. | | | | | | | | |

^{b}p < 0.05 [vs control (HBSS-treated) mice].
^{c}p < 0.05 [vs the same pfu of the appropriate control avipox virus (i.e., 10⁷ avipox(F)-GM-CSF vs. 10⁷ avipox(F)-WT)].
^{d}p < 0.05 [vs 1 injection of rGM-CSF].

The most pronounced increase in the total number of cells/node occurred in mice injected with 10⁸ pfu of either of the GM-CSF-expressing recombinant viruses. Along with the increase in lymph node cellularity was a selective increase in the percentage of class II-expressing cells in mice treated with the recombinant avipox viruses expressing 5 GM-CSF as compared with the control viruses (Table 3). Usually, 25-29% of the lymph node cells from untreated mice or mice injected with either avipox-WT or avipox-RG express MHC class II antigens. Injection of (10⁷ or 10⁸ pfu) of either of the recombinant avipox-GM-CSF viruses increased that percentage to 43-51% by day 7, with an accompanying 3-fold boost in their MFI (Table 3). When the regional lymph nodes from the avipox-GM-CSF-treated mice were analyzed 21 days after injection, a sustained increase in the percentage of class II⁺ lymph node cells was found. Multiple injections with recGM-CSF were needed to enhance lymph node cellularity and MHC class II expression (Table 3). Upon cessation of recGM-CSF treatment, the changes in lymph node cellularity and class 11 expression quickly return to pretreatment levels, and as shown in Table 3, by day 21.

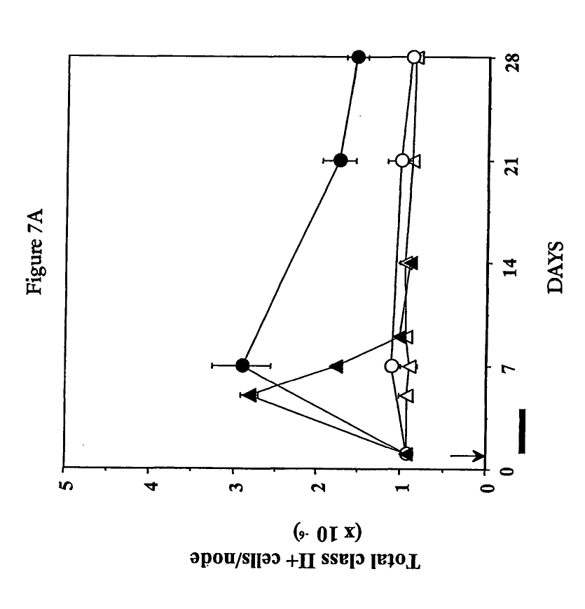

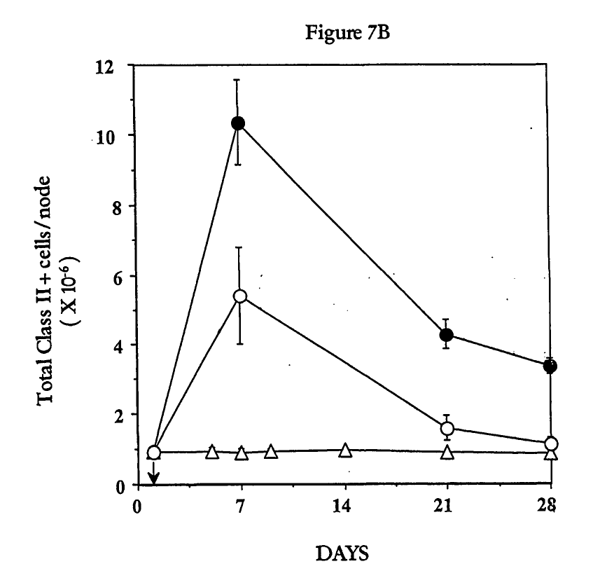

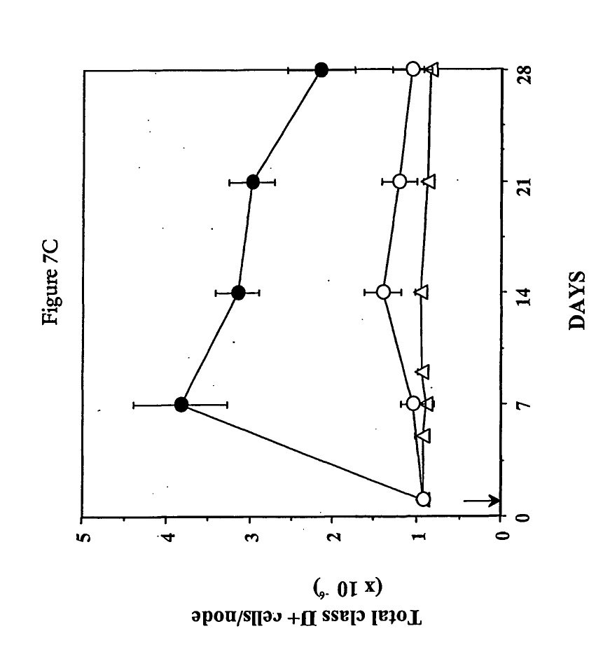

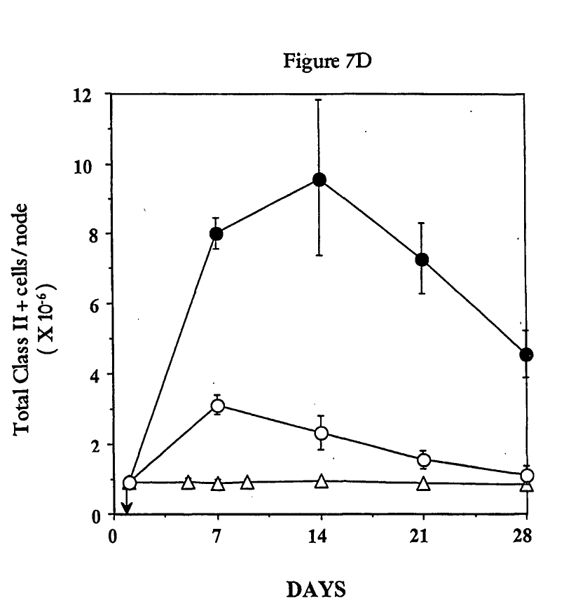

A more in-depth examination of the time course of the increased class II-expressing lymph node cells in mice given a single injection of avipox(F)-GM-CSF or avipox(A)-GM-CSF was carried out. As summarized in FIG. 7, mice were injected with 10⁷ or 10⁸ pfu either recombinant avipox virus or the appropriate control viruses and the total number of class II-expressing cells in the regional lymph nodes were determined at weekly intervals. Elevated levels in the total number of class II⁺ cells/lymph node were observed in mice injected with 10⁷ or 10⁸ pfu of either recombinant avipox-GM-CSF virus by day 7 (FIG. 7). The absolute number of class II-expressing cells in mice treated with 10⁸ pfu of either recombinant avipox-GM-CSF virus remained elevated for 21-28 days. A comparative time course for the changes in the total number of class II-expressing lymph node cells in mice treated for 4 days with recGM-CSF is presented (FIG. 7A, dashed line).

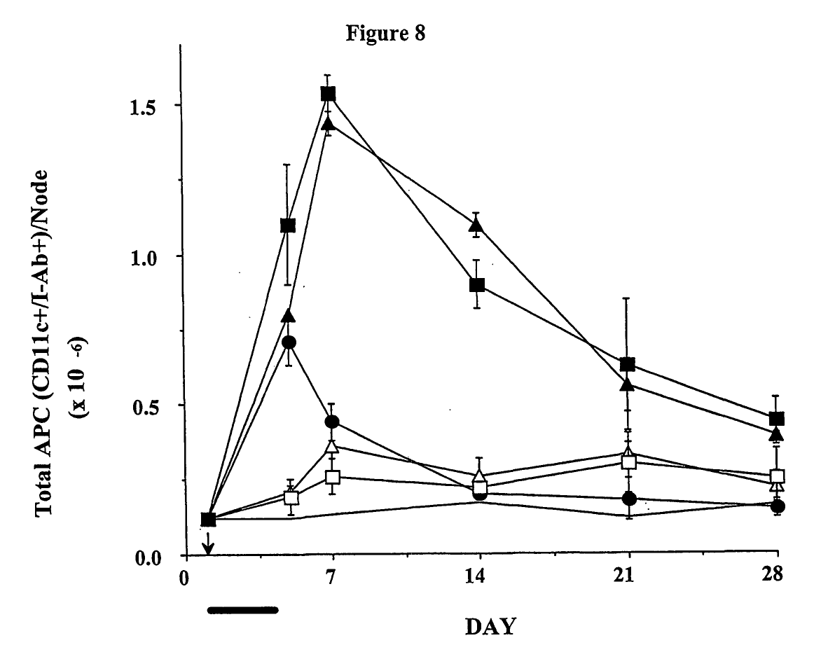

The increase in class II expression levels in the regional lymph nodes has been reported to be comprised of higher class II levels on B cells and an influx of CD11c⁺/I-Ab⁺ cells (21). The CD11c⁺/I-Ab⁺ cells were also CD3⁻, CD19⁻, Ter119⁻, NK1.1⁻, CD11b⁺, DEC205⁺, CD80⁺ and CD86⁺, a cell-surface phenotype profile consistent with that of APC, particularly macrophages and dendritic cells (37). FIG. 8 summarizes the temporal changes in the APC population in the regional lymph nodes isolated from mice treated with 10⁸ pfu of either recombinant avipox-GM-CSF virus or the appropriate control viruses. Approximately 1-2% of lymph node cells from untreated mice were APC as defined by their antigen phenotype. Seven days after the injection of 10⁸ pfu of either avipox-GM-CSF that percentage was increased approximately 3-fold (data,not shown). The absolute number of CD11 c⁺/class II⁺ cells in the nodes on day 7 after the injection of either recombinant avipox-GM-CSF virus was increased 12-fold (FIG. 8). In addition, the time course for the increase in the number of APC/node was virtually identical to that for the total number of class II-expressing cells. Lymph nodes from avipox-WT and -RG treated mice did not contain higher numbers of APC (FIG. 8). Those nodes did contain significantly higher number of B and T cells.

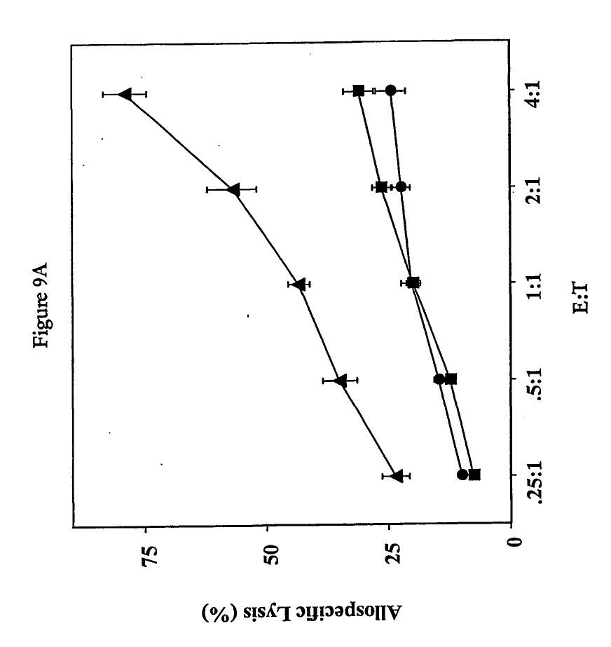

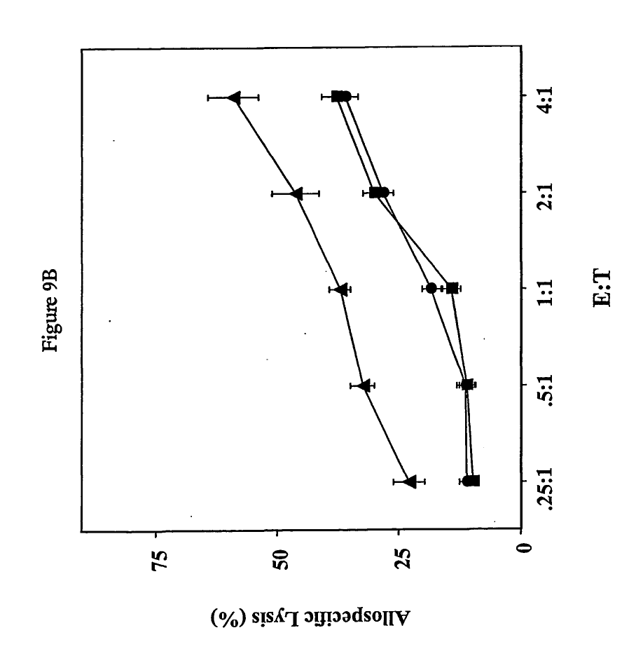

Regional nodes from B6 mice injected with avipox-GM-CSF or the control virus were isolated after 7 and 21 days and used to generate alloreactive CTL in vitro from mixed lymphocyte cultures (FIG. 9). When tested for allospecific lytic activity, lymph node cells isolated from avipox-GM-CSF-treated mice on day 7 (FIG. 9A) and 21 (FIG. 9B) were significantly (p<0.05) more potent than lymph node cells from either untreated or mice treated with the control avipox virus.

### EXAMPLE 5

### Effects of Multiple Avipox-GM-CSF Injections

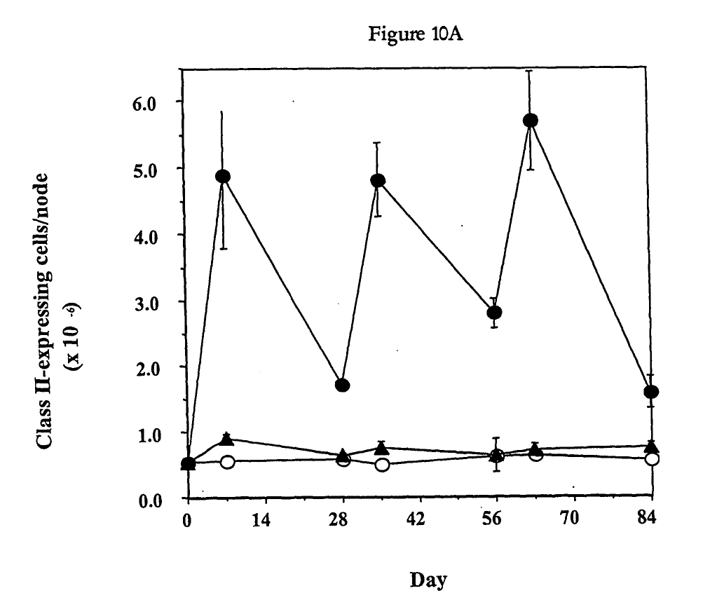

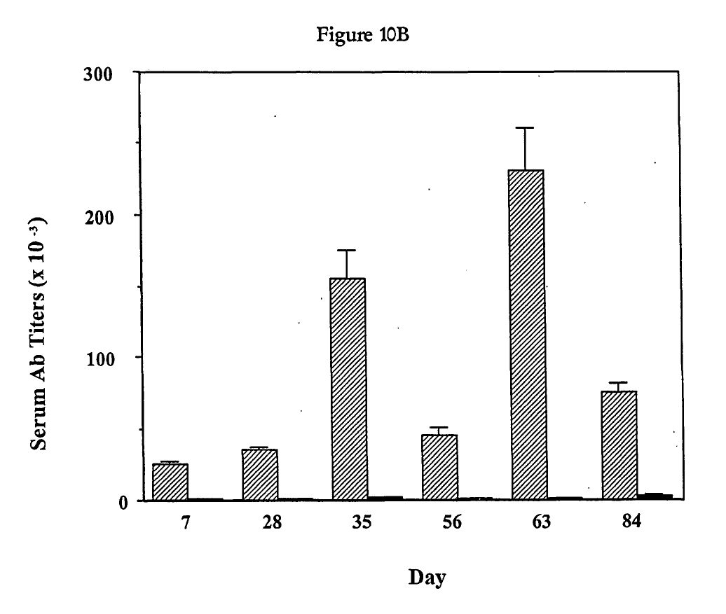

Studies were carried out in which mice received 3 monthly injections of avipox-GM-CSF and the regional lymph nodes examined for changes in total class II-expressing cells following each injection. Serum samples were also analyzed for the development of anti-avipox and anti-GM-CSF antibody titers. Seven days after the initial avipox-GM-CSF injection, the absolute number of class II cells was increased approximately 10-fold—from 0.5 to 4.9×10⁶/lymph node (FIG. 10A). By day 28, that number had fallen to 1.7×10⁶, but after the second injection of avipox-GM-CSF on day 28, rose to 4.8×10⁶ by day 35. A third injection of avipox-GM-CSF was administered on day 56 once again increased the number of class II⁺ cells/node from 2.8 to 5.7×10⁶. (FIG. 10A). Injection of avipox (A)-RG resulted in no observable change in the number of class II⁺ lymph node cells.

Serum samples were taken on days 7,28, 35, 56, 63 and 84 and analyzed for the presence of anti-avipox and -GM-CSF IgG titers. Measurable anti-avipox antibody titers were observed on days 7 and 28 (FIG. 10B). After the second injection of avipox-GM-CSF, administered on day 28, the serum anti-avipox IgG titers were boosted >100,000. The third injection of avipox-GM-CSF resulted, in yet, another increase of serum anti-avipox IgG titers to >200,000. No detectable serum anti-GM-CSF IgG titers were found at any of the time points (FIG. 10B).

### EXAMPLE 6

### Adjuvant Effects Avipox-GM-CSF on Antigen-Specific Immunity

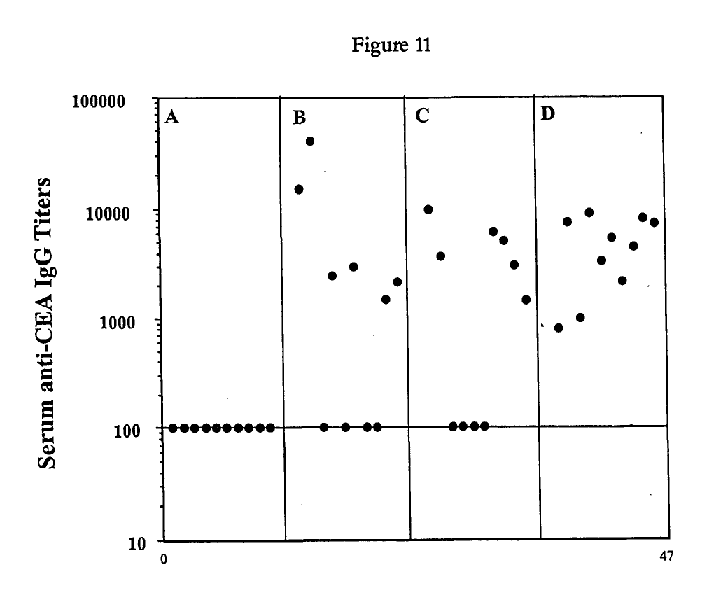

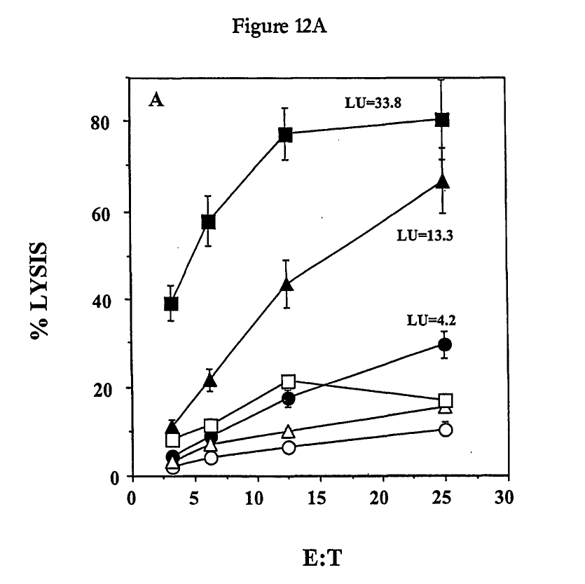

Anti-CEA Antibody Responses in CEA.Tg Mice. CEA.Tg mice were vaccinated twice at monthly intervals with avipox-CEA alone or combined with a either a single injection of avipox-GM-CSF or recGM-CSF administered for 4 consecutive days. The presence of anti-CEA IgG serum titers in 60% of the mice vaccinated with avipox-CEA alone or avipox-CEA combined with recGM-CSF (FIGS. 11B and 11C). All 10 CEA.Tg mice vaccinated with avipox-CEA and avipox-GM-CSF (FIG. 11, panel D) developed anti-CEA IgG responses. No CEA antibody titers were detected in the sera of naive or CEA.Tg mice vaccinated with the control virus (avipox-RG)(FIG. 11, panel A).

T-cell Proliferative Responses to CEA. Primary CEA-specific splenic T-cell proliferative responses were used to evaluate the effectiveness of delivering GM-CSF in a recombinant avipox virus versus the use of multiple GM-CSF injections (Table 2). CEA-specific T cell proliferation was measured by [³H]thymidine incorporation following a five-day incubation of splenic T cells isolated from vaccinated CEA.Tg mice. CEA-specific T cell proliferation was demonstrated by the inability (i) of OVA to stimulate T cell proliferation and (ii) of splenic T cells isolated from mice immunized with a control avipox virus to proliferate in the presence of soluble CEA (Table 2). When avipox-CEA was administered in combination with avipox-GM-CSF or recGM-CSF, the resultant splenic T cell proliferative response to soluble CEA was boosted (P<0.05) No CEA-specific lymphoproliferation was found using splenic T cells isolated from avipox-RG-vaccinated CEA.Tg mice. (Table 4).

TABLE 4
³H-Thymidine incorporation by splenic T cells isolated from nonimmune and immune CEA.Tg mice.

| | | Avipox(A)- ± GM-CSF (cpm ± SEM) | | | | | |
|---|---|---|---|---|---|---|---|
| | | Avipox(A)-CEA | | | Avipox(A)-RG | | |
| Ag | | | | +Avipox(A)- | | | +avipox(A)- |
| (μg/ml) | untreated | -recGM-CSF | +recGM-CSF | GM-CSF | -recGM-CSF | +recGM-CSF | GM-CSF |
| CEA (50) | 597 ± 196^{b} | 6,569 ± 790 | 12,321 ± 1149^{c} | 18,113 ± 332^{c,d} | 1602 ± 144 | neg | neg |
| (25) | 176 ± 71^{b} | 4,831 ± 271 | 8,625 ± 165^{c} | 14,034 ± 547^{c,d} | 1375 ± 88 | neg | neg |
| (12.5) | neg | 3,182 ± 106 | 5,470 ± 493^{c} | 10,964 ± 436^{c,d} | 1501 ± 243 | neg | neg |
| (6.25) | neg | 1,752 ± 97 | 2,524 ± 417^{c} | 10,445 ± 419^{c,d} | 2929 ± 87 | neg | neg |
| OVA (50) | neg | 1,691 ± 67 | 2,190 ± 83^{c} | 2,369 ± 522^{c} | 1045 ± 93 | neg | 1122 ± 83 |
| Con A (12.5) | 212,096 | 194,400 | 214,516 | 234,987 | 197,036 | 234,987 | 212,890 |
| ^{a}CEA.Tg mice (2-3/group) were administered 10⁸ pfu of avipox(A)-CEA or avipox(A)-RG s.c. (100 μl) 2x at monthly intervals. GM-CSF was administered as a recombinant protein or in a recombinant avipox(A) virus as described in the Materials and Methods. Four-6 weeks after the second immunization, mice were sacrificed, splenic T cells isolated and pooled according to treatment group. the T cell proliferative | | | | | | | |
| # responses to soluble CEA, OVA and Con A were measured by ³H-tymidine incorporation. | | | | | | | |

^{b}Data are presented as the delta cpm [minus cpm (2621-7973) in wells containing T cells, APC, and no antigen) ± SEM from a representative experiment. For the Con A-stimulated wells, the average cpm is shown (SEM < 10%). Three separate experiments were performed with similar results. neg = cpm < media control.
^{c}p < 0.05 (vs. Avipox(A)-CEA-immune mice).
^{d}p < 0.05 (vs. Avipox(A)-CEA + recGM-CSF-treated mice).

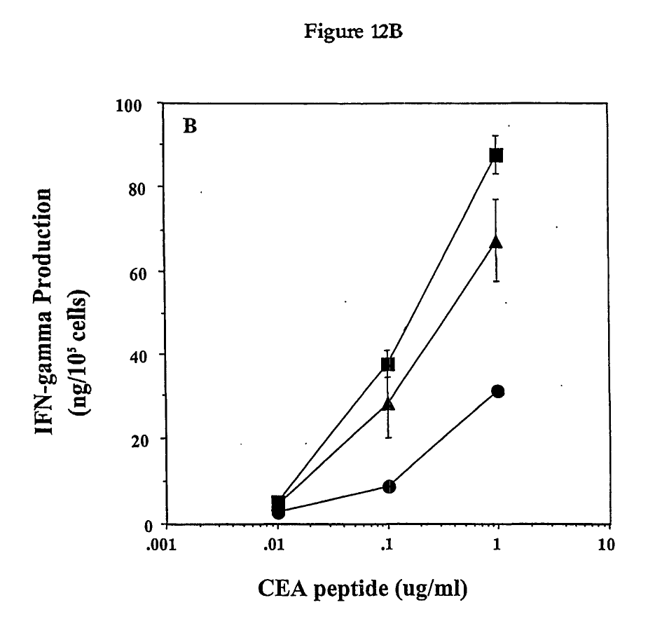

CEA peptide-specific T-Cell Lysis. Since repeated attempts to detected primary peptide-specific CTL responses in vaccinated CEA-Tg mice failed (data not shown), splenic T cells were isolated from immune CEA.Tg mice and subsequently stimulated in vitro in the presence of an 8-mer peptide spanning CEA amino acids 526-533 and IL-2. T cell proliferation in response to CEA₅₂₆₋₅₃₃, IL-2 and irradiated APC was observed in those CEA-Tg mice immunized with avipox-CEA alone or in combination with avipox-GM-CSF or recGM-CSF. After the two in vitro stimulations, >90% of the isolated T cells from the three cell populations were CD8⁺. Moreover, those T cells were capable of killing syngeneic (EL-4) targets pulsed with the CEA₅₂₆₋₅₃₃ peptide (FIG. 12A). CEA peptide-specific EL-4 lysis was highest (p<0.05 vs. either avipox-CEA or avipox-CEA+recGM-CSF-immunized mice), as measured by lytic units, for the T cell line that was obtained from CEA.Tg mice vaccinated with avipox-CEA in combination with avipox-GM-CSF (FIG. 12A). When the EL-4 target cells were pulsed with an irrelevant peptide (i.e., Flu NP), background levels of cytolysis were observed (FIG. 12A). T cell lines generated from CEA.Tg mice vaccinated with avipox-CEA combined with either avipox- or recGM-CSF also produced higher gamma-interferon levels than the T cell lines generated from CEA.Tg mice vaccinated with avipox-CEA alone (FIG. 12B). No IL-4 was found in any of those cultures.

### EXAMPLE 7

### Antitumor Immunity

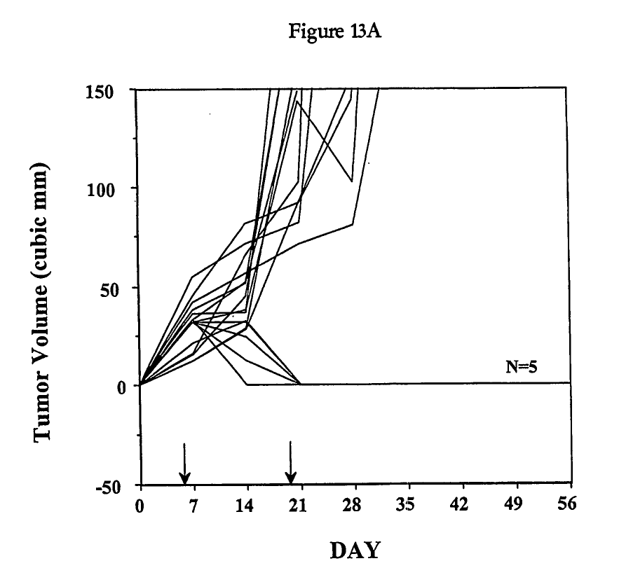

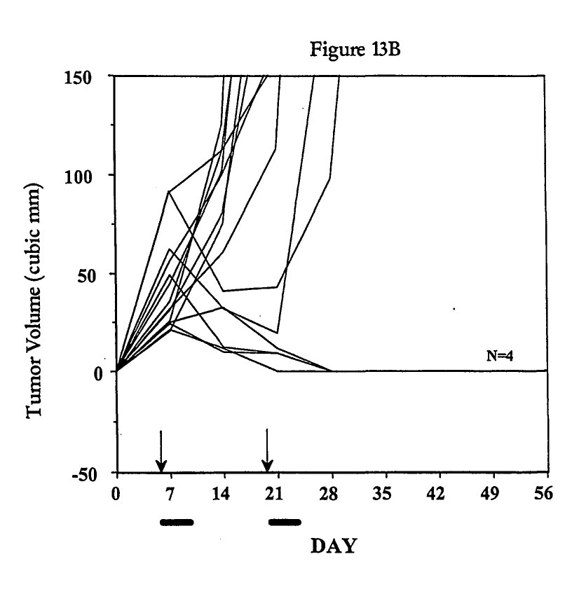

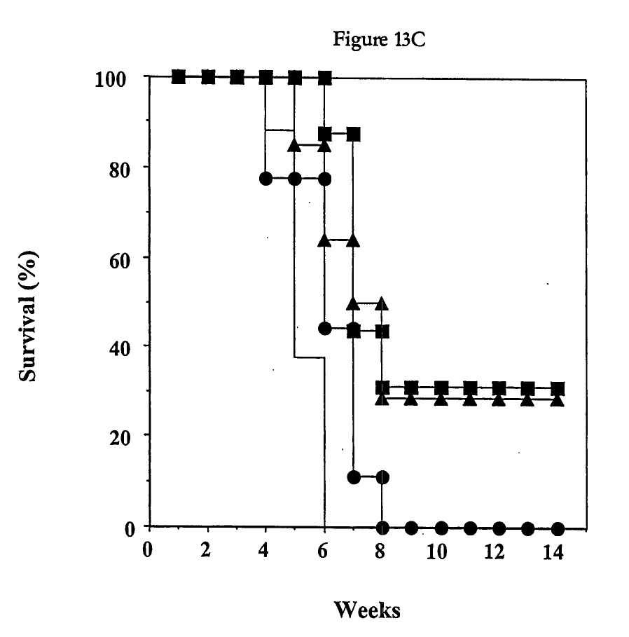

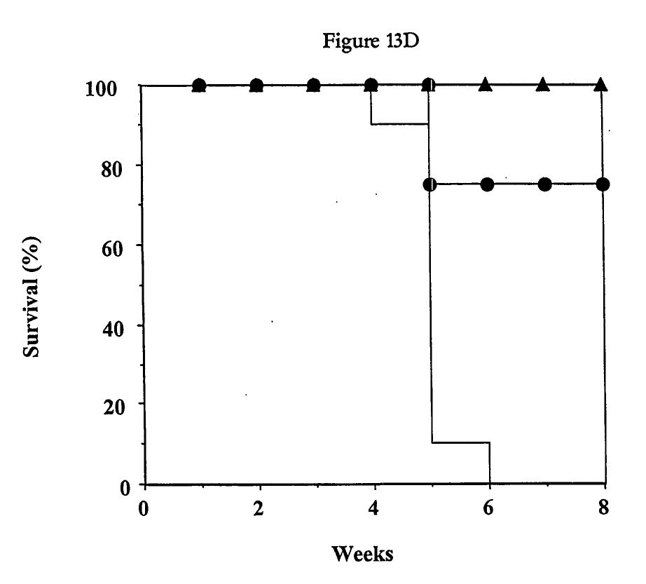

CEA.Tg mice bearing MC-38-CEA-2 tumors were vaccinated with avipox-CEA alone or in combination with avipox-GM-CSF or rGM-CSF as well as the control virus, avipox-RG alone, or combined with GM-CSF. MC-38-CEA-2 tumors grow progessively in naive CEA.Tg mice and mice that were vaccinated with avipox-RG alone or in combination with GM-CSF, and those mice were sacrificed 6-7 weeks after tumor inoculation (Table 3). Avipox-CEA vaccination resulted in a transient slowing of tumor growth in some CEA.Tg mice; however, survival was not prolonged (FIG. 13C).

Vaccination with avipox-CEA combined with avipox-GM-CSF induced measurable reductions in tumor volume of 6 of 16 CEA.Tg mice (FIG. 13A). By day 35, the average tumor volume of the avipox-CEA+avipox-GM-CSF treatment group was significantly smaller (P<0.05) than that of untreated, avipox-RG+avipox (A)-GM-CSF or avipox-CEA-vaccinated CEA.Tg mice. In fact, five tumor-bearing CEA.Tg mice vaccinated with avipox-CEA and avipox-GM-CSF became tumor free (Table 3, FIG. 13A) by day 28 and remained so for 14 weeks (FIG. 13C). At that time, the five tumor-free CEA.Tg mice were challenged with MC-38-CEA-2 tumor cells, and all were protected (FIG. 13D). Four of 14 CEA.Tg mice vaccinated with avipox-CEA and rGM-CSF also became tumor free (Table 3; FIG. 13B), and three of those four mice rejected tumor at challenge (FIG. 13D).

TABLE 5
Immunotherapy of tumor-bearing CEA.Tg mice.

| | | | Tumor Growth | | |
|---|---|---|---|---|---|
| | | | Tumor Volume | | |
| | | | (Mean ± SEM | Tumor | |
| Immunogen^{a} | # mice | # Died | @ day 35) | Regression | Tumor Eradication |
| None | 8 | none | 2186.0 ± 386.9 | none | none |
| avipox(A)-CEA | 9 | none | 1511.1 ± 287.1^{b} | 1 | none |
| avipox(A)-CEA + avipox(A)- | 16 | 1 | 371.5 ± 134.3^{b} | 6^{c} | 5^{c} |
| GM-CSF | | | | | |
| avipox(A)-CEA + recGM-CSF | 14 | none | 622.1 ± 201.6^{b} | 6^{c} | 4^{c} |
| avipox(A)-RG | 8 | none | 1921.2 ± 333.5 | none | none |
| avipox(A)-RG + avipox(A)- | 9 | none | 1716.6 ± 412.2 | none | none |
| GM-CSF | | | | | |
| avipox(A)-RG + rec-GM-CSF | 5 | none | 1663.2 ± 505.2 | none | none |

^{a}CEA.Tg mice were immunized with the approximate avipox recombinants ± either avipox-GM-CSF or recGM-CSF at two week intervals as described in the Materials and Methods. Data were compiled from two separate experiments with the exception of the avipox(A)-RG + recGM-CSF group which represents data from a single experiment.
^{b}p < 0.5 (vs. control CEA.Tg mice).
^{c}p < 0.05 (vs. avipox(A)-CEA vaccinated CEA.Tg mice).

### EXAMPLE 8

### rF-GM-CSF Enhances CEA-Specific T-Cell Responses to CEA Vaccines In Vivo

### Methods

Female C57BL/6 mice were vaccinated with 1 time with 1×10⁸ pfu/mouse with avipox(F)WT, rF-CEA, or rF-CEA/TRICOM, as disclosed herein and in Cancer Research 59:5800-5807, 1999. One half of each group received 1×10⁷ pfu/mouse avipox (F)-GM-CSF as adjuvant to measure if GM-CSF would enhance T-cell response, while the remaining mice did not receive avipox (F)-GM-CSF (n=3 mice/group). Fourteen days later, splenocytes from vaccinated groups were collected for analysis of cellular immune responses. To quantitate T-cell responses, T cells from vaccinated mice were incubated with irradiated splenocytes in the presence of several concentrations of CEA protein for 5 days. T cells were also incubated with Con A or ovalbumin for positive and negative proliferation controls. During the final 18 hours of incubation, ³H-Thymidine was added to measure T-cell proliferation.

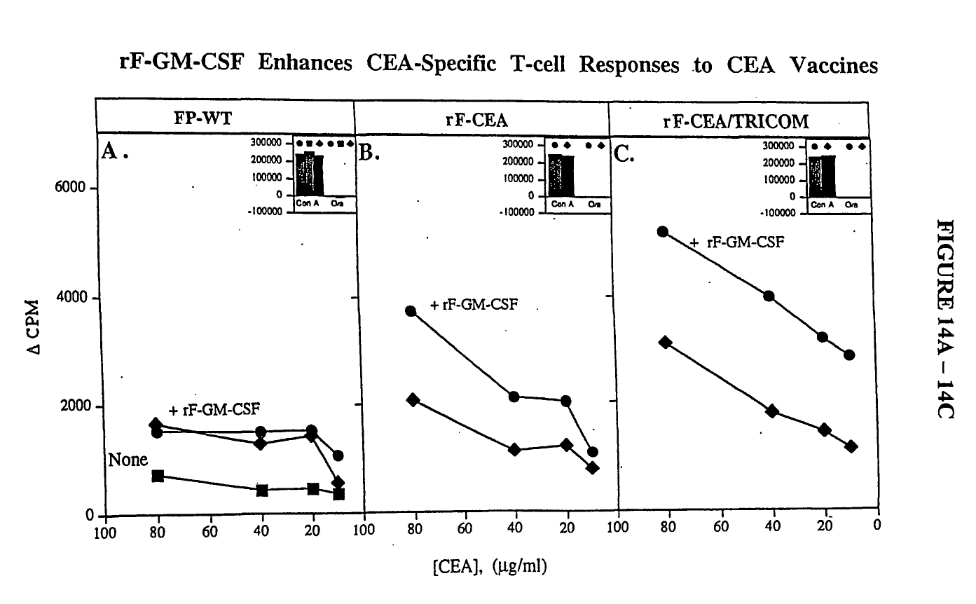

These experiments demonstrate that avipox (F)-GM-CSF, when given in combination with rF-CEA, or rF-CEA/TRICOM enhances the ability of these vectors to activate CEA-specific T-cell responses in vivo (FIGS. 14A-14C).

### EXAMPLE 9

### CD4 Response to β-Galactosidase: Immunoadjuvant Effects of Fowlpox-GM-CSF

### Methods

Lymphoproliferative responses to β-gal by splenocytes isolated from mice immunized with β-gal combined with incomplete Freunds adjuvant with or without Fp-mu-GM-CSF were determined. Mice were initially vaccinated with 100 μg β-gal combined with incomplete Freunds adjuvant (triangles) (mixed in a 1:1 per volume ratio) or adjuvant alone (circles). In selected groups, either Fp-mu-GM-CSF (10⁷ pfu) (diamonds) or Fp-WT (10⁷ pfu) (squares) was added with the immunogen and injected s.c. Thirty days after the vaccination spleens were removed and the T-cells isolated and used in a lymphoproliferative assay which included β-gal protein (100-6.25 ug/ml) and 5×10⁵ irradiated antigen-presenting cells isolated from naive C57BL/6 mice. ³H-Thymidine was added after 5 days of in vitro culture and the amount incorporated was measured 24 h later.

### Results

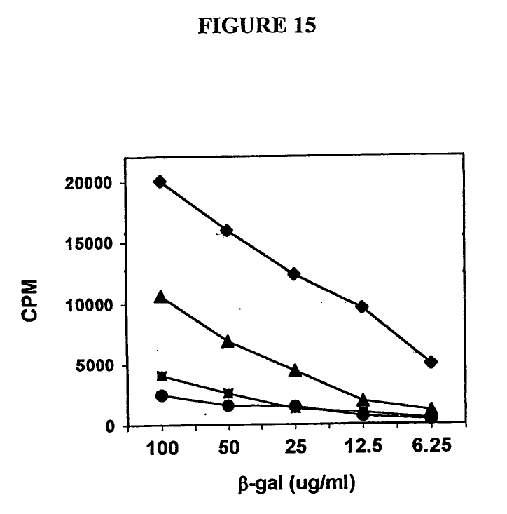

The date demonstrated that the recombinant avipox virus (Fowlpox) expressing murine GM-CSF substantially augments host cellular (i.e., CD4) immune responses when a whole protein (β-galactosidase) is used as an immunogen (FIG. 15).

### EXAMPLE 10

### Intravesical Administration of Avipox-F-GM-CSF to Patients with Bladder Cancer

Avipox-GM-CSF is administered intravesically to patients with bladder cancer. Patients are administered between 10⁶ and 10¹¹ pfu of avipox-GM-CSF via a catheter to infect bladder carcinoma cells. Avipox-GM-CSF is administered from 1 to 10 times at intervals of 1 day, 1 week, or 1 month. Efficacy of treatment is evaluated clinically.

### EXAMPLE 11

### Direct Intratumor Injection of Avipox-GM-CSF in Patients With Head and Neck Carcinoma

Avipox-GM-CSF is directly injected into tumors such as head and neck, melanoma and breast metastasis of the skin. Between 10⁵ and 10⁹ pfu of avipox-GM-CSF is administered from one to 10 times at daily, weekly, or monthly intervals.

### EXAMPLE 12

### Vaccination of Patients with CEA-Expressing Carcinomas

Avipox-GM-CSF is used in combination with an avipox-CEA-TRICOM vaccine to treat any CEA expressing tumor. Avipox-CEA-TRICOM is a vaccine in which the fowlpox recombinant expresses the tumor antigen CEA and three different costimulatory molecules: B7-1, ICAM-1 and LFA-3. The avipox-GM-CSF is given at doses of 10⁶ to 10¹⁰ pfu/injection. The avipox-GM-CSF is administered either before (1 day to 1 week), at the same time of or after (1 day to 1 week) administration of avipox-CEA-TRICOM. The avipox-CEA-TRICOM is given at a dose of 10⁶ to 10¹⁰ pfu/injection.

### EXAMPLE 13

### Avipox-GM-CSF in Combination with Recombinant Poxvirus Expressing HIV and SIV Antigens in SIV and SHIV Challenge Models in Rhesus Macaques

The avipox-GM-CSF is given at doses of 10⁶ to 10¹⁰ pfu/injection subcutaneously. The avipox-GM-CSF is administered either before (1 day to 1 week), at the same time of, or after administration (1 day to 1 week) of avipox-HIV antigen-TRICOM, avipox-SIV antigen-TRICOM, avipox-HIV antigen-B7, or avipox-SIV antigen-B7 subcutaneously at 10⁶ to 10¹⁰ pfu/injection.

### Discussion

GM-CSF is believed to act as a potent biological adjuvant for vaccines by its ability to attract professional APC to a local injection site which then migrate into the regional lymph nodes to mediate host immune responses (15, 21, 22). Previous studies (15) in which recombinant GM-CSF protein was injected for 4-5 consecutive days, reported an enrichment of the regional lymph nodes with class II-expressing APC which has, in turn, been correlated with a boost in host immunity. Different vehicles have been used to deliver GM-CSF to an immunization site. Some of those approaches include the introduction of the GM-CSF gene via retroviral vectors into tumor cell vaccines (19, 20), fusion proteins (18) and replication-deficient (25) recombinant poxviruses. In the present study, replication-defective recombinant avipox [fowlpox, canarypox (ALVAC)] viruses expressing GM-CSF were given as single s.c injection to B6 mice. The resultant increases in the absolute number of lymph node cells (Table 3), the percentage (Table 3), MFI (Table 3) and absolute number of class II-expressing cells (FIGS. 7A-7D) and the number of CD11c⁺/I-Ab⁺ cells (FIG. 8) within the regional draining lymph nodes were all consistent with the elaboration of biologically active GM-CSF by the recombinant avipox viruses. Two different recombinant avipox viruses, fowlpox and canarypox (ALVAC), expressing GM-CSF were compared and no apparent differences were observed.

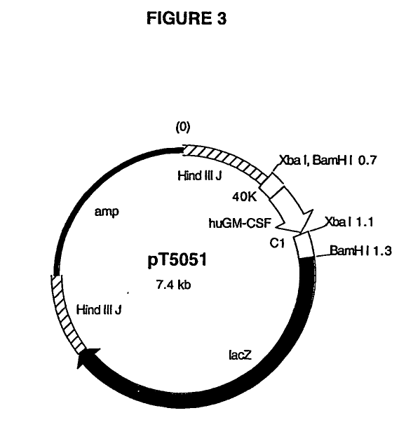

The use of recombinant avipox viruses to deliver GM-CSF to an immunization site may have several advantages over using recGM-CSF. The magnitude of the increase in the absolute number of CD11c⁺/I-Ab⁺ cells in the regional lymph nodes was much greater in mice injected with the avipox-GM-CSF viruses than with recGM-CSF. For example, after 4-5 days of recGM-CSF treatment, the number of CD11c⁺I-Ab⁺ lymph node cells was increased by approximately 6-fold when compared with untreated mice (0.71 vs. 0.12×10⁶/node, FIG. 3). A single injection of either recombinant avipox-GM-CSF virus 10⁸ pfu boosted the absolute number of CD11c⁺/I-Ab⁺ lymph node cells by almost 70-fold (1.44 vs. 0.12×10⁶/node, FIG. 3). The second advantage of using recombinant avipox viruses to deliver GM-CSF may be the temporal changes associated with the enrichment of APC within the regional nodes. As shown in FIG. 8, recGM-CSF needs to be administered for 4-5 days to increase APC concentration within the regional nodes. Upon cessation of recGM-CSF treatment, the changes within the injection site rapidly disappear (approx. 4-5 days). On the other hand, the elevations in the absolute number of class II⁺ and CD11 c⁺/I-Ab⁺ lymph node cells were sustained in the regional lymph nodes of mice injected with either recombinant avipox-GM-CSF virus for 21-28 days (FIG. 8). In fact, lymph nodes cells isolated 21 days after avipox(A)-GM-CSF injection generated a more robust allospecific CTL response in vitro, indicating their functional integrity (FIG. 9B). One might argue that the recombinant avipox-GM-CSF viruses produce a depot of GM-CSF after injection and the prolonged changes seen in the regional node would represent the slow release of the cytokine. That seems unlikely since the in vivo half-life of GM-CSF is on the order of 2-3 days. While not being bound by theory, a more plausible explanation is that the replication-defective avipox viruses remain at the injection site and continuously produces GM-CSF which, in turn, mediates the sustained changes seen in the regional nodes.

If these recombinant avipox viruses are to be used to deliver biologically active GM-CSF to a vaccination site, they must be compatible with certain anticancer vaccines. To test that hypothesis, recombinant avipox-GM-CSF viruses as well as recGM-CSF were evaluated for their abilities to augment CEA-specific host immunity in CEA.Tg mice when using avipox-CEA as a tumor vaccine. Vaccination of CEA.Tg mice with avipox-CEA or, as previously reported, a recombinant vaccinia-CEA virus (29), induces CEA-specific humoral and cell-mediated immunity. However, the CEA-specific immunity generated in CEA.Tg mice vaccinated with a recombinant poxvirus-CEA vaccine was relatively weak. Indeed, in the present study, avipox-CEA vaccination induced a transient growth inhibition of CEA-expressing subcutaneous tumors in the CEA.Tg mice (FIG. 13B). Incorporating GM-CSF, either as a recombinant avipox virus or recombinant protein, increased the CEA-specific CD4⁺-proliferative (Table 4) and CD8⁺-mediated-lytic (FIG. 12A) responses in avipox-CEA-vaccinated CEA.Tg mice. In fact, the anti-CEA-specific cellular immune responses were significantly more potent in those CEA.Tg mice in which avipox-GM-CSF, not recGM-CSF, was the biological vaccine adjuvant. Thus, it seems that recombinant avipox viruses expressing a tumor antigen and GM-CSF are compatible and can be injected simultaneously. Moreover, if the recombinant avipox-CEA virus produces CEA continuously for 21-28 days, then the co-existence of antigen with elevated local GM-CSF levels might result in a continuous loading of dendritic cells with tumor antigen.

While that may explain the improved cellular response to CEA, one is left to speculate why those changes did not mediate more potent antitumor responses in the CEA.Tg mice vaccinated with avipox-CEA and avipox-GM-CSF. One possible explanation is that the use of an experimental model in which the cell-mediated immunity is generated against a self antigen may introduce host/tumor factors that would counterbalance the antitumor response.

Because of their ability to infect and express gene products as well as their documented safety in clinical trials (38-42), recombinant avipox viruses are attractive candidates for cancer vaccines. Previous exposure to vaccinia does not alter the immune response to recombinant avipox viruses (43) and in diversified prime-and-boost protocols the two viruses induce antitumor immunity in murine models (36). The present findings expand the use of recombinant avipox viruses to include GM-CSF delivery to enrich an immunization site with APC, thereby, augmenting the generation of antigen-specific antitumor immunity. Another finding was the ability of avipox(A)-GM-CSF to enrich the regional lymph nodes with APC after repeated injections. That was accomplished despite the presence of anti-avipox serum antibody titers which have been observed in these and other studies (24, 44). Ea fact, in a recent clinical trial, multiple injections of avipox-CEA administered to patients with advanced CEA-positive tumors led to an ongoing increase in the CEA-specific T cell precursor frequencies. A third advantage of using a recombinant avipox-GM-CSF virus would be the ease of mixing it with an immunogen, such as avipox-CEA, and administering the vaccine as a single injection as compared with 4-5 daily injections of recGM-CSF. That would simplify vaccine design, reduce treatment costs, while, possibly, maximizing the adjuvant effects of GM-CSF.

### References

1. Witmer-Pack M. D., Olivier, W., Valinsky, I., Schuler, G., and Steinman, R. M. Granulocyte-macrophage colony-stimulating factor is essential for the viability and function of cultured murine epidermal Langerhans cells. J. Exp. Med., 166: 1484-1498, 1987.

2. Heufler, C., Koch, F., and Schuler, G. Granulocyte-macrophage colony-stimulating factor and interleukin-1 mediate the maturation of murine epidermal Langerhans cells into potent immunostimulatory dendritic cells. J. Exp. Med., 167: 700-705, 1988.

3. Romani, N., Koide, S., Crowley M, Witmer-Pack, M., Livingston, A. M., Fathman, C. G., Inaba, K., and Syeinman, R. M. Presentation of exogenous protein antigens by dendritic cells to T-cell clones. J. Exp. Med., 169: 1169-1178, 1989.

4. Morrissey, P. J., Bressler, L., Park, L. S., Alpert, A., and Gillis, S. Granulocyte-macrophage colony-stimulating factor augments the primary antibody response by enhancing the function of antigen presenting cells. J. Immunol., 139: 1113-1119, 1987.

5. Disis, M. L., Bernhard, H., Shiota, F. M., Hand, S. L., Gralow, J. R., Huseby, E. S., Gillis, S., and Cheever, M. A. Granulocyte-macrophage colony-stimulating factor: An effective adjuvant for protein and peptide-based vaccines. Blood, 88: 202-210, 1996.

6. Jager, E., Ringhoffer, M., Dienes, H. P., Arand, M., Karbach, J., Jager, D., Ilemann, C., Hagedom, M., Oesch, F, and Knuth, A. Granulocyte-macrophage colony-stimulating factor enhances immune responses to melanoma-associated peptides in vivo. Int. J. Cancer, 67: 54-62, 1996.

7. Chen, T. T., Tao, M -H., and Levy, R. Idiotype-cytokine fusion proteins as cancer vaccines. Relative efficiency of IL-2, IL-4, and granulocyte-macrophage colony-stimulating factor. J. Immunol., 153: 4775-4787, 1994.

8. Kwak, L. W., Young, H. A., Pennington, R. W., and Weeks, S. D. Vaccination with syngeneic, lymphoma-derived immunoglobulin idiotype combined with granulocyte-macrophage colony-stimulating factor primes mice for a protective T-cell response. Proc. Natl. Acad Sci, 93: 10972-10977, 1996.

9. Samanci, A., Yi, Q., Fagerberg, J., Strigard, K., Smith, G., Ruden, U., Wahren, B. and Mellstedt, H. Pharmacological administration of granulocytelmacrophage-clony-stimulating factor is of significant importance for the induction of a strong humoral and cellular response in patients immunized with recombinant carcinoembryonic antigen. Cancer. Immunol. Immunother., 47: 131-147, 1998.

10. Ragnhammer, P., Fagerberg, J., Frodin, J -E., Wersall, P., Hansson, L -O., and Mellstedt, H. Granulocyte-macrophage colony-stimulating factor augments the induction of antibodies, especially anti-idiotype antibodies, to therapeutic monoclonal antibodies. Cancer Immunol. Immunother., 40: 367-375, 1995.

11. Jager, E., Ringhoffer, M., Dienes, H. P., Arand, M., Karbech, J., Jager, D., Ilsemann, C., Hagedom, M., Oesch, F., and Knuth, A. Granulocyte-macrophage colony-stimulating factor enhances immune responses to melanoma-associated peptides in vivo. Int. J. Cancer, 67: 54-62, 1996.

12. Tarr, P. E., Lin, R., Mueller, E. A., Kovarik, J. M., Guillaume, M., Jones, T. C. Evaluation of tolerability and antibody response after recombinant human granulocyte-macrophage colony-stimulating factor (rhGM-CSF) and a single dose of recombinant hepatitis B vaccine. Vaccine, 14: 1199-1204, 1996.

13. Leong, S. P. L., Enders-Zohr, P., Zhou, Y -M., Stuntebeck, S., Habib, F. A., Allen, Jr., R. E., Sagebiel, R. W., Glassberg, A. B., Lowenberg, D. W. and Hayes, F. A. Recombinant granulocyte-macrophage colony-stimulating factor (rhGM-CSF) and autologous melanoma vaccine mediate tumor regression in patients with metastatic melanoma. J. Immunother., 22: 166-174, 1999.

14. Bendandi, M., Gocke, C. D., Kobrin, C. B., Benko, F. A., Stemas, L. A., Pennington, R., Watson, T. M., Reynolds, C. W., Gause, B. L., Duffey, P. L., Jaffe, E. S., Creekmore, S. P., Longo, D. L. and Kwak, L. W. Complete molecular remissions induced by patient-specific vaccination plus granulocyte-macrophage colony-stimulating factor against lymphomas. Nature Med., 5: 1171-1177, 1999.

15. Disis, M. L., Bernhard, H., Shiota, F M., Hand, S. L., Gralow, J. R., Huseby, E. S., Gillis, S. and Cheever, M. A. Granulocyte-macrophage colony-stimulating factor: An effective adjuvant for protein and peptide-based vaccines. Blood, 88: 202-210, 1996.

16. Weiss, W. R., Ishii, K. J., Hedstrom, R. C., Sedegah, M., Ichino, M., Bamlhart, K., Klinman, D. M., and Hoffman, S. L. A plasmid encoding murine granulocyte-macrophage colony-stimulating factor increases protection conferred by a malaria DNA vaccine. J. Immunol., 161: 2325-2332, 1998.

17. Lee, S. W., Cho, J. H., and Sung, Y. C. Optimal induction of hepatitis C virus envelope-specific immunity by bicistronic plasmid DNA inoculation with the granulocyte-macrophage colony-stimulating factor gene. J. Virol., 72: 8430-8436, 1998.

18. Chen, T. T., Tao. M -H., and Levy, R. Idiotype-cytokine fusion proteins as cancer vaccines. Relative efficiency of IL-2, IL-4, and granulocyte-macrophage colony-stimulating factor. J. Immunol., 153: 4775-4787, 1994.

19. Dranoff, G., Jaffee, E., Lazenby, A., Golumbek, P., Levitsky, H., Brose, K., Jackson, V., Hamada, H., Pardoll, D., and Mulligan, R. C. Vaccination with irradiated tumor cells engineered to secrete murine granulocyte-macrophage colony-stimulating factor stimulates potent, specific, and long-lasting anti-tumor immunity. Proc. Natl. Acad. Sci., 90: 3539-3543, 1993.

20. Vieweg, J., Rosenthal, F. M., Bannedji, R., Heston, W. D. W., Fair, W. R., Gansbacher, B., and Gilboa, E. Immunotherapy of prostate cancer in the Dunning rat model: Use of cytokine gene modified vaccines. Cancer Res., 54: 1760-1765, 1994.

21. Kass, E., Parker, J., Schlom, J. and Greiner, J. W. Comparative Studies of the Effects of recombinant GM-CSF and GM-CSF administered via a poxvirus to enhance the concentration of antigen presenting cells in regional lymph nodes. Cytokine, 2000 (in press).

22. Kielian, T., Nagai, E., Ikubo, A., Rasmussen, C. A., and Suzuki, T. Granulocyte/macrophage-colony-stimulating factor released by adenovirally transduced CT26 cells leads to the local expresion of macrophage inflammatory protein 1α and accumulation of dendritic cells at vaccination sites in vivo. Cancer Immunol. Immunother., 48:123-131, 1999.

23. Issekutz, T. B. Characteristics of lymphoblasts appearing in efferent lymph on response to immunization with vaccinia virus. Immunology, 56: 23-31, 1985.

24. Leong, K. H., Ramsey, A. J., Boyle, D. B. and Ranshaw, I. A. Selective expression of immune responses by cytokines coexpressed in recombinant fowlpox virus. J. Virol., 68: 8125-8130, 1994.

25. Puisieux, I., Odin, L., Poujol, D. Moingeon, P., Tartaglia, J., Cox, W. and Favrot, M. Canarypox virus-mediated interleukin 12 gene transfer into murine mammary adenocarcinoma induces tumor suppression and long-term antitumoral immunity. Human Gene Ther., 91: 2481-2492, 1998.

26. Gold, P. and Freedman, S. O. Demonstration of tumor-specific antigens in human colonic carcinomata by immunological tolerance and absorption techniques. J. Exp. Med., 121: 439-462, 1965.

27. Shuster, J., Thomson, D. M. P., Fuks, A. and Gold, P. Immunologic approaches to diagnosis of malignancy. Prog. Exp. Tumor Res., 25: 89-139, 1980.

28. Hasegawa, T., Isobe, K, Tsuchiya, Y., Oikawa, S., Nakazato, H., Ikezawa, H., Nakashimna, 1. and Shimokata, K Establishment and characterization of human carcinoembryonic antigen transgenic mice. Br. J. Cancer, 64: 710-714, 1991.

29. Clarke, P., Mann, J., Simpson, J. F., Rickard-Dickson, K. and Primus, F. J. Mice transgenic for human carcinoembryonic antigen as a model for immunotherapy. Cancer Res., 58: 1469-1477, 1998.

30. Eades-Pemer, A -M., van der Putten, H., Hirth, A., Thompson, J., Neumaier, M., von Kleist, S., and Zimmermann, W. Mice transgenic for the human carcinoembryonic antigen gene maintain its spatiotemporal expression pattern. Cancer Res., 54: 4169-4176, 1994.

31. Kass, E., Schlom, J., Thompson, J., Guadagni, F., Graziano, P. and Greiner, J. Induction of protective host immunity to carcinoembryonic antigen (CEA), a self antigen in CEA transgenic mice, by immunizing with a recombinant vaccinia-CEA virus. Cancer Res., 591: 676-683, 1999.

32. Robbins, P. F., Kantor, J., Salgaller, M., Horan Hand. P., Fernsten, P. D., Schlom J. Transduction and expression of the human carcinoembryonic antigen (CEA) gene in a murine colon carcinoma cell line. Cancer Res., 51: 3757-3762, 1991.

33. Muraro, R, Wunderlich, D., Thor, A., Lundy, J., Noguchi, P., Cunningham, R., and Schlom, J. Definition of monoclonal antibodies of a repertoire of epitopes on carcinoembryonic antigen differentially expressed in human colon carcinoma versus normal adult tissues. Cancer Res., 45: 5769-5780, 1985.

34. Dexter, T. M., Garland, J., Scott, D., Scolnick, E., and Metcalf, D. Growth of factor-dependent hemopoietic precursor cell lines. J. Exp. Med., 52: 1036-1047, 1980.

35. Fries, L. F., Tartaglia, J., Taylor, J., Kauffman, E. F., Maignier, B., Paoletti, E. and Plotkin, S. Human safety and immunogenicity of a canarypox-rabies glycoprotein recombinant vaccine: an alternative poxvirus vector system. Vaccine, 14: 428-434, 1996.

36. Hodge, J. W., McLaughlin, J. P., Kantor, J. A. and Schlom, J. Diversified prime and boost protocols using recombinant vaccinia virus and recombinant non-replicating avian pox virus to elicit T-cell immunity and antitumor responses. Vaccine, 15: 759-768, 1997.

37. Maraskovsky, E., Brasel, K., Teepe, M., Roux, E. R., Lyman, S. D., Shortman, K., and McKenna, H. J. Dramatic increase in the numbers of functionally mature dendritic cells in Flt3 ligand-treated mice: Multiple dendritic cell subpopulations identified. J. Exp. Med., 184: 1953-1962, 1996.

38. Wang, M., Bronte, V., Chen, P. W., Gritz, L., Panicali, D., Rosenberg, S. A., and Restifo, N. Active immunotherapy of cancer with a nonreplicating recombinant fowlpox virus encoding a model tumor-associated antigen. J. Immunol., 154: 4685-4692, 1995.

39. Roth, J., Dittmer, D., Rea, D., Tartaglia, J., Paoletti, E., and Levine, A J. p53 as a target for cancer vaccines: recombinant canarypox virus expressing p53 protect mice against lethal tumor cell challenge. Proc. Natl. Acad. Sci., 93: 4781-4786, 1996.

40. Marshall, J. L., Hawkins, M J, Tsang, K. Y., Richmond, E., Pedicano, J. E., Zhu, M -Z. and Schlom, J. Phase I study in cancer patients of a replication-defective avipox recombinant vaccine that expresses human carcinoembryonic antigen. J. Clin. Oncol. 17: 332-337, 1999.

41. Cadoz, M., Strady, A., Meignier, B., Taylor, J., Tartaglia, J., Paoletti, E. and Plotkin, S. Immunisation with canarypox virus expressing rabies glycoprotein. Lancet, 339: 1429-1432, 1992.

42. Taylor, J., Weinberg, R, Tartaglia, J., Richardson, C., Alkhatib, G., Briedis, D., Appel, M., Norton, E. and Paoletti, E. Nonreplicating viral vectors as potential vaccines: Recombinant canarypox virus expressing measles virus fusion (F) and hemagglutinin (HA) glycoproteins. Virology, 187: 321-328, 1992.

43. Uppal, P. K. and Nilakantan, P. R. Studies on the serological relationships between avian pox, sheep pox, goat pox and vaccinia viruses. J. Hyg. Camp., 68: 349-358, 1970.

44. Kawakita, M., Rao, G. S., Ritchey, J. K., Ornstein, D. K, Hudson, M I. A., Tartaglia, J., Paoletti, E., Humphrey, P. A., Harmon, T. J. and Ratliff T. L. Effect of carcarypox vrus (ALVAC)-mediated cytokine expression on murine prostate tumor growth. J. Natl. Cancer Inst., 891:428-436, 1997.

45. McLaughlin, J. P., Abrams, S., Kantor, J., Dobrzanski, M. J., Greenbaum, J., Schlom, J. and Greiner, J. Immunization with a syngeneic tumor infected with recombinant vaccinia virus expressing granulocyte-macrophage colony-stimulating factor (GM-CSF) induces tumor regressin and long-lasting systemic immunity. J. of Immunotherapy 20(6):449-459, 1997.

46. Chakrabarti, S., Brechling, K. and Moss, B. (1985) Mol. Cell. Biol. 5:3403-3409.

47. Gritz, L., Destree, A., Gormier, N., Day, E., Stallard, V., Caiazzo, T. Mazzara, G. and Panicali D. (1990) J. Virol. 64:5948-5957.

49. Jenkins, S., Gritz, L., Fedor, C., O'Neil, E., Cohen, L. and Panicali, D. (1991) AIDS Research and Human Retroviruses 7:991-998.

50. Mayr, A., Hochstein-Mihntzel, V., and Sticki, H. (1975) Infection 3:6-14.

51. Mazzara, G., Destree, A, and Mahr, A. (1993) Meth. Enzymol. 217:557-581.

52. Sambrook, J., Fritsch, E. F., and Maniatis, T., eds, Molecular Cloning Cold Spring Harbor Laboratory, 1989.

53. Sutter, G., Wyatt, S. S., Foley, P. I., Bennink, J. R. and Moss, B. (1994) Vaccine 12:1032-1040.

We claim:

1. A pharmaceutical composition comprising a replication-defective virus encoding granulocyte-monocyte-colony stimulating factor (GM-CSF) and a pharmaceutically acceptable carrier.

2. The pharmaceutical composition according to claim 1, wherein the replication-defective virus encodes human GM-CSF.

3. The pharmaceutical composition according to claim 1, wherein the virus is produced using a plasmid vector designated pT5052 deposited with the American Type Culture Collection under Accession No. PTA-2099.

4. The pharmaceutical composition according to claim 1, wherein the replication-defective virus is a poxvirus.

5. The pharmaceutical composition according to claim 1 wherein the replication-defective virus is an avipox virus.

6. The pharmaceutical composition according to claim 5 wherein the avipox virus is selected from the group consisting of fowlpox virus, canarypox virus, MVA, and derivatives thereof.

7. The pharmaceutical composition according to any one of claims 1-6, wherein the replication-defective virus further encodes at least one antigen or immunological epitope thereof.

8. The pharmaceutical composition according to claim 7, wherein the antigen is selected from the group consisting of tumor specific antigen, tumor associated antigen, tissue-specific antigen, bacterial antigen, viral antigen, yeast antigen, fungal antigen, protozoan antigen, parasite antigen and mitogen.

9. The pharmaceutical composition according to claim 7, wherein the immunological epitope comprises at least one amino acid sequence selected from the group consisting of SEQ ID NO: 1 through SEQ ID NO: 36 and combinations thereof.

10. The pharmaceutical composition according to claim 1 further comprising a vector encoding at least one costimulatory molecule.

11. The pharmaceutical composition according to claim 10, wherein the costimulatory molecule is B7.1.

12. The pharmaceutical composition according to claim 10, wherein the costimulatory molecule is B7.1/ULA-3/ICAM-1.

13. The pharmaceutical composition according to any one of claims 1-12, further comprising a vector encoding alpha interferon, beta interferon, or gamma interferon.

14. The pharmaceutical composition according to any one of claims 1-12 further comprising a vector encoding at least one cytokine.

15. The pharmaceutical composition according to claim 14, wherein the cytokine is IL-12.

16. The pharmaceutical composition according to any one of claims 1-6, further comprising an Fc receptor-directed bispecific antibody.

17. The pharmaceutical composition according to claim 16, wherein the Fc receptor is selected from the group consisting of FcγRI (CD64), FcγRII (CD32) and FcγRIII (CD16).

18. The pharmaceutical composition according to claim 16, wherein the antibody is an anti-CD3-directed antibody.

19. The pharmaceutical composition according to any one of claims 16-18, wherein the bispecific antibody has tumor-directed specificity.

20. The pharmaceutical composition according to claim 19, wherein the specificity is selected from the group consisting of HER-2/neu, EGF-receptor, CD15 antigen and a EpCAM molecule.

21. The pharmaceutical composition according to claims 1-16, further comprising at least one antigen or immunological epitope source, and optionally a conventional adjuvant.

22. The pharmaceutical composition according to claim 21, wherein the conventional adjuvant is selected from the group consisting of Ribi Detox™, alum, QS-21, Freund's complete adjuvant, and Freund's incomplete adjuvant.

23. The pharmaceutical composition according to claim 21, wherein the antigen or immunological epitope source is a protein, peptide, antibody, anti-idiotypic antibody, lipid, carbohydrate, cell, cell extract, cell fragment, DNA encoding an antigen, or encoding an immunological epitope thereof RNA encoding an antigen or encoding an immunological epitope thereof, or a vector encoding at least one antigen or immunological epitope thereof.

24. The composition according to claim 21, wherein the antigen is selected from the group consisting of a tumor specific antigen, tumor associated antigen, tissue-specific antigen, bacterial antigen, viral antigen, yeast antigen, fungal antigen, protozoan antigen, parasite antigen and mitogen.

25. The composition according to claim 21, wherein the immunological epitope comprises at least one amino acid sequence selected from the group consisting of SEQ ID NO: 1 through SEQ ID NO: 36, and combinations thereof.

26. The composition according to claim 24, wherein the bacterial antigen is derived from a bacterium selected from the group consisting of Chlamydia, Mycobacteria, Legionella, Meningiococcus, Group A Streptococcus, Hemophilus influenzae, Salmonella, and Listeria

27. The composition according to claim 24, wherein the viral antigen is derived from a virus selected from the group consisting of Lentivirus, retrovirus, Herpes virus, Hepatitis virus, Orthomyxovirus and Papillomavirus.

28. The composition according to claim 27, wherein the Lentivirus is HIV-1 or HIV-2.

29. The composition according to claim 27, wherein the Herpes virus is HSV or CMV.

30. The composition according to claim 27, wherein the Hepatitis virus is Hepatitis A, Hepatitis B, Hepatitis C, Hepatitis D or Hepatitis E.

31. The composition according to claim 27, wherein the orthomyxovirus is influenza virus.

32. The composition according to claim 24, wherein the tumor associated antigen, tumor specific antigen or tissue-specific antigen is selected from the group consisting of CEA, MART-1, MAGE-1, MAGE-3, GP-100, MUC-1, MUC-2, point mutated ras oncogene, normal or point mutated p53, overexpressed p53, CA-125, PSA, PSMA, C-erb/132, BRCA I, BRCA II, PSMA, tyrosinase, TRP-1, TRP-2, NY-ESO-1, TAG72, KSA, HER-2/neu, bcr-abl, pax3-fkhr, ews-fli-1, modified TAAs, splice variants of TAAs, functional epitopes and epitope agonists thereof.

33. The composition according to claim 24, wherein the yeast or fungal antigen is derived from a yeast or fungus selected from the group consisting of Aspergillus, Nocardia, Histoplasmosis, Candida, and Cryptosporidia.

34. The composition according to claim 24, wherein the parasitic antigen is derived from a Plasmodium species, Toxoplasma gondii, Pneumocystis carinii, Trypasosoma species, or Leishmania species.

35. The composition according to claim 23, wherein the vector encoding an antigen or immunological epitope thereof is selected from the group consisting of poxvirus, adenovirus, Herpes virus, alphavirus, picomavirus, iridovirus, DNA plasmids, and RNA.

36. The composition according to claim 35, wherein the poxvirus is orthopox, avipox, capripox or suipox.

37. The composition according to claim 35, wherein the antigen is selected from the group consisting of a tumor specific antigen, tumor associated antigen, tissue-specific antigen, bacterial antigen, viral antigen, yeast antigen, fungal antigen, protozoan antigen, parasite antigen, and mitogen.

38. The composition according to claim 35, wherein the immunological epitope comprises at least one amino acid sequence selected from the group consisting of SEQ ID NO: 1 through SEQ ID NO: 36, and combinations thereof.

39. The composition according to claim 23, wherein the antigen or immunological epitope source is a vector encoding at least one antigen or antigenic epitope thereof.

40. The composition according to claim 23, wherein the vector encodes at least one costimulatory molecule selected from the group consisting of B7-1, B7-2, ICAM-1, LFA-3, 4-1BBL, CD59, CD40, CD70, OX-40L, VCAM-1, mammalian homologs thereof and combinations thereof.

41. The composition according to claim 40, wherein the costimulatory molecule is B7.1.

42. The compositon according to claim 40, wherein the costimulatory molecules are at least B7-1, ICAM-1 and LFA-3.

43. The composition according to claim 40, wherein the recombinant replication-defective virus encoding GM-CSF is avipox, the antigen source is a vector encoding CEA or epitope thereof, and the costimulatory molecule is B7 or B7.1/LFA-3/ICAM-1.

44. The composition according to any one of claims 1-15, further comprising a cytokine, chemokine or Flt-3L.

45. The composition according to any one of claims 1-15, further comprising at least one antibiotic, antifungal agent, anti-viral agent or combinations thereof.

46. The composition according to any one of claims 1-6, further comprising erythropoietin.

47. The composition according to claim 46, wherein the erythropoietin is recombinantly produced.

48. A host cell infected, transfected or induced with the recombinant replication-defective virus encoding GM-CSF according to any of claims 1-15 and 21-47.

49. The host cell according to claim 48, wherein the host cell is an antigen presenting cell or precursor thereof, a premalignant cell, a hyperplastic cell, tumor cell, or a tumor cell fused to an antigen presenting cell.

50. The host cell according to claim 49, wherein the antigen presenting cell is a dendritic cell or precursor or derivative thereof, a monocyte, macrophage, B-cell, fibroblast or muscle cell.

51. The host cell according to any one of claims 49 or 50, wherein the antigen presenting cell is derived from bone marrow, spleen, skin, peripheral blood, tumor, lymph node, or muscle.

52. The host cell according to claim 50, wherein the derivative is a TNF α-treated dendritic cell, a CD40-treated dendritic cell, or a subpopulation of adherent cells.

53. A dendritic cell or precursor thereof comprising a foreign nucleic acid sequence encoding GM-CSF provided by a recombinant replication-defective virus.

54. A tumor cell or precursor thereof comprising a foreign nucleic acid sequence encoding GM-CSF provided by a recombinant replication-defective virus.

55. The cell according to claims 53 or 54, wherein the cell further comprises a foreign nucleic acid sequence encoding at least one costimulatory molecule.

56. The cell according to claim 55, wherein the costimulatory molecules are selected from the group consisting of B7-1, B7-2, ICAM-1, LFA-3, 4-1BBL, CD59, CD40, CD70, OX-40L, VCAM-1, mammalian homologs thereof and combinations thereof.

57. The cells according to claims 56, wherein the costimulatory molecule is B7.1.

58. The cells according to claim 56, wherein the multiple costimulatory molecules are at least B7-1, ICAM-1 and LFA-3.

59. The cells according to any one of claims 53-58 further comprising a foreign nucleic acid sequence encoding at least one target antigen or immunological epitope thereof.

60. The cells according to claim 59, wherein the foreign nucleic acid sequence encoding at least one target antigen or immunological epitope thereof is provided by a recombinant vector, RNA or DNA from a tumor cell lysate, or by fusion with a tumor cell comprising said sequence.

61. The cells according to any one of claims 59 or 60, wherein the target antigen or immunological epitope thereof is selected from the group consisting of a tumor specific antigen, tumor associated antigen, tissue-specific antigen, bacterial antigen, viral antigen, yeast antigen, fungal antigen, protozoan antigen, parasite antigen and mitogen.

62. The cells according to any one of claims 59 or 60, wherein the immunological epitope comprises at least one amino acid sequence selected from the group consisting of SEQ ID NO: 1 through SEQ ID NO: 36, and combinations thereof.

63. A pharmaceutical composition comprising the cells according to any one of claims 53-62, and optionally an exogenous source of target antigen or immunological epitope thereof.

64. A method of enhancing an immune response in an individual comprising administration of the composition according to any one of claims 1-15 and 21-47 in an amount sufficient to enhance the immune response.

65. The method according to claim 64, wherein a route of administration is intravenous, subcutaneous, intralymphatic, intratumoral, intradermal, intramuscular, intraperitoneal, intrarectal, intravaginal, intranasal, oral, via bladder instillation, intranasal, intraarterial, intravesical or via scarification.

66. The method according to claim 64, wherein the enhancement is migration of antigen presenting cells at an injection site, regional lymph node at a tumor site or combination thereof.

67. The method according to claim 66, wherein the antigen presenting cells express CD11c⁺/I-Ab⁺, MHC Class II, or combination thereof.

68. The method according to claim 66, wherein the enhancement is of antigen presenting cell proliferation, function or combination thereof.

69. The method according to claim 64, wherein the enhanced immune response is a cell mediated or humoral response.

70. The method according to claim 64, wherein the enhancement is of CD4⁺ T cell activation, CD8⁺ T cell activation, or combination thereof.

71. The method according to claim 64; wherein the enhancement is in IL-2 production, IFN-γ production, TNF-α production, or combinations thereof.

72. A method of enhancing an antigen-specific T-cell response in an individual to a target antigen or immunological epitope thereof comprising administering a recombinant replication-defective poxvirus encoding GM-CSF in combination with a recombinant virus comprising a nucleic acid sequence encoding a target antigen or immunological epitope thereof and optionally also comprising a foreign nucleic acid sequence encoding at least one B7 molecule, a foreign nucleic acid sequence encoding ICAM-1, and a nucleic acid sequence encoding LFA-3, in an amount effective to enhance at least one T-cell response.

73. The method according to claim 72, wherein the enhancement is of CD4⁺ T cell activation, CD8⁺ T cell activation, or combination thereof.

74. The method according to claim 72, wherein the enhancement is in IL2 production, IFN-γ production or combination thereof.

75. The method according to claim 72, wherein the enhancement is of antigen-specific cytotoxicity.

76. A method of enhancing an anti-tumor response in an individual with a tumor comprising administration of the composition according to claims 1-15 or 21-47 in an amount effective to enhance the anti-tumor response.

77. The method according to claim 76, further comprising administration of a target antigen or immunological epitope thereof, cell expressing a target antigen or immunological epitope thereof, or cells pulsed with a target antigen or immunological epitope thereof.

78. The method according to any one of claim 76 or 77, wherein the composition is directly injected in situ into a tumor or adjacent to a tumor.

79. The method according to claim 78, wherein the tumor is a head tumor, neck tumor, melanoma, breast tumor, pancreatic tumor, prostate tumor, colorectal tumor, or metastatic tumor.

80. The method according to claim 79, wherein the tumor is a metastatic breast skin lesion.

81. The method according to any one of claims 76-79, wherein the composition is injected during surgery.

82. The method according to claim 81, wherein the tumor is a colonrectal cancer or pancreatic cancer.

83. The method according to any one of claims 76-79, wherein the composition is injected into a lymph node distal to or draining a tumor site.

84. The method according to any one of claims 76-79, further comprising the administration of activated, target antigen specific lymphocytes.

85. The method according to any one of claims 76-79, wherein the anti-tumor response is tumor regression, increase in disease-free interval, or increase in survival.

86. A method of enhancing an immune response in an individual comprising administration of a cell according to any of claims 48-62 in an amount effective to enhance an immune response.

87. A method of enhancing an immune response in an individual comprising administration of a tumor cell, or precursors thereof according to claim 49 or 54 in an amount effective to enhance an immune response.

88. The method according to any one of claims 86 or 87, wherein the cells are autologous, syngeneic or allogeneic with the individual.

89. The method according to any one of claims 86 or 87, wherein the cells have been pulsed with a target antigen or epitope thereof.

90. The method according to any one of claims 86 or 87, further comprising the administration of a target cell, target antigen or immunological epitope thereof.

91. The method according to any one of claims 86-90, further comprising the administration of activated, target antigen specific lymphocytes.

92. A method of enhancing an immune response to an antigen or immunological epitope thereof in an individual comprising administration of a first recombinant vector encoding GM-CSF followed by administration of a second recombinant vector encoding GM-CSF wherein at least one recombinant vector is a replication-defective virus.

93. The method according to claim 92, wherein the replication defective virus is selected from the group consisting of poxvirus, herpes virus, adenovirus and adeno-associated virus.

94. The method according to claim 93, wherein the poxvirus is selected from the group consisting of fowlpox, canary pox and a Modified Vaccinia Ankara strain.

95. The method according to claim 92, wherein a second recombinant vector is replication competent.

96. The method according to claim 95, wherein the second vector is vaccinia.

97. A method of treating neutropenia in an individual comprising administration of a recombinant replication-defective virus encoding GM-CSF in an amount effective to treat the neutropenia.

98. The method according to claim 97, further comprising the administration of an antibiotic, antifungal agent, antiparasite agent, or antiviral agent.

99. The method according to claim 97, wherein the neutropenia is resultant from chemotherapy, corticosteroid therapy, irradiation, or an infection.

100. The method according to any one of claims 97-99, wherein a dose raises the neutrophil count to normal levels.

101. A method for treating cytopenias in patients with myeloidysplastic syndrome comprising administration of a recombinant replication-defective virus encoding GM-CSF in combination with erythropoietin in an amount effective to treat the cytopenia.

102. The method according to claim 101, wherein a dose in the range of about 10⁵ to about 10¹⁰ pfu of the recombinant replication-defective virus encoding GM-CSF is administered at a weekly or monthly interval.

103. The method according to any one of claims 101 or 102, wherein a dose in the range of about 150 to about 300 u/k of erythropoietin is administered.

104. The method according to any one of claims 101-104, wherein the dose is provided on alternate days.

105. The method according to any one of claims 101-104, wherein the treatment increases the neutrophil count and erythroid precursors.

106. A method of enhancing an antitumor immune response comprising administration of a replication-defective virus encoding GM-CSF in combination with a bispecific antibody.

107. The method according to claim 106, wherein the bispecific antibody has tumor-directed specificity.

108. The method according to claim 107, wherein the bispecific antibody has a specificity selected from the group consisting of HER-2/neu, EGF-receptor, CD15 antigen and a EpCAM molecule.

109. The method according to any one of claims 107-108, wherein the bispecific antibody is directed to a tumor cell epitope and a cytotoxin trigger molecule.

110. A method of enhancing an immune response to a vaccination comprising administration of a replication-defective virus encoding GM-CSF at the vaccination site or regional lymph node in an amount effective to enhance the immune response to a vaccine.

111. The method according to claim 110, wherein the vaccine comprises at least one tumor antigen or tumor associated antigen.

112. The method according to claim 110, wherein the vaccine is selected from the group consisting of DPT vaccine, Td vaccine, DtaP vaccine, Hib vaccine, DtaP-Hib vaccine, MMR vaccine, Hepatitis A vaccine, Hepatitis B vaccine, Lyme's disease vaccine, influenza vaccine, tetravalent meningococcal polysaccharide, pneumococcal polysaccharide vaccine, anthrax vaccine, cholera vaccine, plague vaccine, yellow fever vaccine and Bacillus Calmette-Guerin vaccine.

113. An immunological adjuvant comprising a replication-defective virus encoding granulocyte-monocyte-colony stimulating factor.

114. A plasmid vector encoding human granulocyte-monocyte colony stimulating factor deposited with the American Type Culture Collection under Accession No. PTA-2099.
